(19) United States
(12) Patent Application Publication
(10) Pub. No.: US 2008/0139545 A1
(43) Pub. Date: Jun. 12, 2008

(54) FORMULATION TO TREAT EAR INFECTION

(76) Inventors: Won-Taek Choe, New York, NY (US); Joseph Baxter Roberson, Palo Alto, CA (US); Michael T. Murray, Mountain View, CA (US)

Correspondence Address: SCHWEGMAN, LUNDBERG & WOESSNER, P.A., P.O. BOX 2938, MINNEAPOLIS, MN, 55402, US

(21) Appl. No.: 11/752,087

(22) Filed: May 22, 2007

Related U.S. Application Data
(60) Provisional application No. 60/747,889, filed on May 22, 2006.

(51) Int. Cl. A61K 31/5383; A61K 31/497; A61P 31/04; A61P 31/12; A61P 31/00

(52) U.S. Cl. 514/230.2; 514/253.08

(57) ABSTRACT

The present invention provides a pharmaceutical composition that includes: (a) at least one of a non-aminoglycoside antibiotic and an anti-inflammatory agent; and (b) a biofilm-dissolving agent. The present invention also provides for methods of killing or inhibiting the growth of a fungus by contacting the fungus with a composition of the present invention, methods of killing or inhibiting the growth of a virus by contacting the virus with a composition of the present invention, methods of killing or inhibiting the growth of a bacteria by contacting the bacteria with a composition of the present invention, methods of treating a disorder in a mammal by administering the composition of the present invention to the mammal, methods for preserving contact lens by contacting the contact lens with a composition of the present invention, methods for cleansing surgical or dental instrument by contacting the fungus with a composition of the present invention, and kits that include (a) a container that includes the pharmaceutical composition of the present invention, and (b) a drug delivery device.

### PRIORITY OF INVENTION

This non-provisional application claims the benefit of priority under 35 U.S.C. § 119(e) to U.S. Provisional Patent Application Ser. No. 60/747,889, filed May 22, 2006, which is herein incorporated by reference.

### BACKGROUND OF THE INVENTION

Patients with a chronically draining ear presents a particular challenge to the otolaryngologist. Many of these patients do not respond to conventional treatments such as oral or topical antibiotics, or even surgery. As such, additional treatment modalities are always of interest.

N-acetylcysteine is a well known antioxidant and mucolytic used primarily to treat acetaminophen overdose and pulmonary diseases such as cystic fibrosis. It acts as an antioxidant both by scavenging free radicals directly and by replenishing intracellular glutathione stores (Kharazmi et al., Int. J. Immunopharmacol., 10:39 (1988)). This antioxidant property may also translate into an anti-inflammatory effect by limiting the oxidative burst that is characteristic of the inflammatory response. NAC acts as a mucolytic by lysing the disulfide bonds that crosslink mucus glycoproteins into a thick matrix (Sheffner, Ann. N.Y. Acad. Sci., 106:298 (1963)). It may also have a direct antibacterial effect by competitively inhibiting cysteine utilization (Zygmunt et al., J. Med. Chem., 11:623 (1968)). NAC has low oral bioavailability (5-10%) because of its high first pass metabolism through the liver (Holdiness, Clin. Pharmacokinetic, 20:123 (1991). As such, NAC is usually administered as a topical mucolytic. Several studies on pooled middle ear effusions have shown NAC to be a potent mucolytic, effecting rapid viscosity reductions of 21-39% at a concentration of 0.1% (FitzGerald et al., Biomed. Pharmacother., 42:505 (1988); FitzGerald et al., Arch. Otolaryngol. Head Neck Surg., 115:462 (1989); Pearson et al., Lancet., 2(8456):674 (1985)).

Several in vitro and animal models have shown NAC to be a promising adjunct to traditional antibiotic therapy. In a bacterial suspension model, NAC exerted an antibiotic effect when administered alone, killing 95% of Pseudomonas cultures at a concentration of only 0.002%. NAC also showed strong synergy with ticarcillin and carbenicillin, but decreased the efficacy of aminoglycoside antibiotics 2-16 fold (Parry et al., J. Clin. Microbiol., 5(1):58 (1977)). NAC has also shown efficacy against biofilms that are otherwise difficult to eradicate with antibiotics alone. It reduces the adherence of Staphylococcus epidermidis to polystyrene (Perez-Giraldo et al., J. Antimicrob. Chemo., 39:643 (1997)); Streptococcus pneumoniae to human oropharyngeal cells (Riise et al., Respiration., 67:552 (2000)); Bacillus species to stainless steel (Olofsson et al., App. Environ. Microbiol., 71(5):2705 (2005)); Moraxella catarrhalis to human pharyngeal cells (Zheng et al., Microbiol. Immunol., 43(2):107 (1999)); Klebsiella pneumoniae to polystyrene (Pinna et al., Opthalmol., 112(5):883 (2005)); Escherichia coli to polystyrene (Marchese et al., Int. J. Antimicrob. Agents, 22:S95 (2003)); Haemophilus influenzae to mucus (Miyamoto et al., Microb. Pathogen, 21:343 (1996)) and human oropharyngeal cells (Riise et al., Respiration (2000)); and polymicrobial cultures to stainless steel (Olofsson et al., App. Environ. Microbiol., 69(8):4814 (2003)) and tracheoesophageal puncture prostheses (Oosterhof et al., Biofouling, 19(6):347 (2003); Schwandt et al., Acta. Otolaryngol., 124:726 (2004)), at concentrations of 0.003% to 0.8%. These reductions are associated with marked drops of extracellular polysaccharide levels, as well as bacterial viability.

Tympanic membrane scarring is one of the hallmarks of chronic, recurrent ear infections. Topical NAC effectively reduced this scarring in rat and rabbit models of otitis media. Marked reductions of fibroblast proliferation, collagen synthesis, and inflammatory markers were also noted (Oveson et al., Clin. Otolaryngol., 18:400 (1993); Ovesen et al., Clin. Otolaryngol., 17:327 (1992); Ozcan et al., Pharm. Res., 45(1):5 (2002); Ozcan et al., Int. J. Ped. Otorhinolaryngol., 63:179 (2002)).

N-acetylcysteine has demonstrated some utility in human studies of otitis media. It increases tympanostomy tube (TT) longevity, and decreases TT reinsertion, recurrence of otitis media, and need for physician visits when applied topically to children at a concentration of 2% (Oveson et al., Acta. Otolaryngol. Suppl., 543:79 (2000)). In addition, NAC may speed the clearance of thick middle ear effusions when administered via transtympanic iontopheresis (Passali et al., Laryngoscope, 94:802 (1984)). NAC was not co-administered with antibiotics or steroids in either study.

NAC is known to be protective in several models of cochlear outer hair cell (OHC) toxicity. Immunostaining of guinea pig cochleas has shown lower levels of intracellular glutathione in outer and inner hair cells when compared to the stria vascularis and spiral ligament (Usami et al., Brain Res., 743:337 (1996)). As such, OHCs may be more susceptible to oxidative damage and, conversely, recoverable with antioxidants than other components of the cochlea. For instance, hydrogen peroxide-mediated damage to OHCs in a cochlear explant study was completely prevented by co-administration with NAC at 0.017% (Dehne et al., Hear Res., 143:162 (2000)). Multiple studies have shown NAC to protect against oxidative OHC damage from cisplatin or aminoglycosides (Choe et al., Otol. Neurotol., 25(6):910 (2004); Bock et al., Hear Res., 9(3):255 (1983)). To our knowledge, no studies have shown NAC to have an ototoxic effect when administered either topically or systemically.

### SUMMARY OF THE INVENTION

Given the established safety profile and possible utility of NAC as described above, the compositions of the present invention were administered in several challenging patients for whom all other known forms of conventional treatment proved ineffective. These patients responded uniformly to the compositions of the present invention. All treated ears ceased to drain within 1-2 weeks of adequate treatment. No ototoxicity was documented by either patient report or audiometry.

It was surprisingly discovered that the combination of NAC and non-aminoglycoside antibiotics provided a synergistic action that neither can reproduce alone. In particular, it is believed that the combination is particularly effective in reducing the bacterial biofilms that may mediate chronic middle ear, mastoid, and Eustachian tube infections. It is notable that many of these patients displayed negative or unimpressive cultures despite frankly persistent otorrhea. This is one of the hallmarks of biofilm infestation.

The present invention provides a pharmaceutical composition that includes: (a) at least one of a non-aminoglycoside antibiotic and an anti-inflammatory agent; and (b) a biofilm-dissolving agent. Specifically, the composition can include a non-aminoglycoside antibiotic or an anti-inflammatory agent. Alternatively, the composition can include a non-aminoglycoside antibiotic and an anti-inflammatory agent. The non-aminoglycoside antibiotic can be, e.g., a β-lactam antibiotic, a cephalosporin, a macrolide, a miscellaneous antibiotic, a penicillin, a tetracycline, an antifungal, an antimalarial agent, an antituberculosis agent, an antiviral, a leprostatic, a miscellaneous anti-infective, a quinolone, a sulfonamide, a urinary anti-infective, a nasal antibiotic, an ophthalmic antibiotic, an ophthalmic antiviral, an ophthalmic quinolone, an ophthalmic sulfonamide, a skin and mucous membrane antibiotic, a skin and mucous membrane antifungal, a skin and mucous membrane antiviral, a skin and mucous membrane miscellaneous anti-infective, a skin and mucous membrane scabicide and pediculicide, a skin and mucous membrane antineoplast, a nitrofuran, or an oxazolidinone.

Additionally, the anti-inflammatory agent can be, e.g., hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluocinolone acetonide, fluocinonide, flucortisone butylesters, fluocortolone, fluprednidene (fluprednidene) acetate, flurandrenolone, halcinonide, hydrocortisone acetate, hydrocortisone butyrate, methylprednisolone, triamcinolone acetonide, cortisone, cortodoxone, fluocinonide, fludrocortisone, diflorasone diacetate, flurandrenolone, fludrocortisone, diflorasone diacetate, flurandrenolone acetonide, medrysone, amcinafel, amcinonide, betamethasone and the balance of its esters, chlorotrianisene, chlorotrianisene acetate, clocortolone, clescinolone, dichlorisone, difluprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, or any combination thereof.

The biofilm-dissolving agent can be, e.g., a furanone, protease, papain, N-acetylcysteine, mercaptoethanol, ebselen, L- and D-methionine, DNAse, RNAse, trypsin, sodium thiosulfate, glutathione, glutathione diethyl ester, fosfomycin, WR-2721, or any combination thereof.

The composition of the present invention can optionally include a preservative, e.g., benzalkonium chloride, quat-15, parabens, dichlorobenzyl alcohol, ethylene diamine tetraacetic acid (EDTA), disodium ethylene diamine tetraacetic acid (EDTA), formaldehyde, gum benzoin, imidazolidinyl urea, phenyl-mercuric acetate, poly aminopropyl biguanide, propyl gallate, sorbic acid, cresol, chloroacetamide sodium benzoate, chloromethyl-methylisothiazolinone, chloromethyl-methylisothiazolon, chloromethyl-methylisothiazolinone benzalkonium chloride, an octylisothiazolinone benzimidazole-compound, chloromethyl-methylisothiazolinone octylisothiazolinone, o-phenylphenol benzisothiazolinone, o-phenylphenol benzisothiazolinone, benzisothiazolinone, an aliphatic amine of 2-thiopyridineoxide, benzoic acid, editic acid, phenolic acid, benzyl alcohol, isopropyl alcohol, benzenethonium chloride, bronopol, cetrimide, chlorhexidine, chlorobutanol, chlorocresol, phenol, phenoxyethanol, phenyl ethyl alcohol, phenylmercuric acetate, phenylmercuric borate, phenylmercuric nitrate, potassium sorbate, proplyene glycol, sodium benzoate, sodium propionate, thimerosal, grapeseed extract, or a combination thereof.

The composition of the present invention can optionally include one or more of a flavoring agent, fragrance, surfactant, decongestant, vasoconstrictor, acid, base, buffer, and an agent to enhance ciliary motility.

The composition of the present invention may be substantially free of oxygen and may have a pH of about 6.0 to about 7.9 or a pH of about 7.2 to about 7.6.

The composition of the present invention may be sterile.

The present invention also provides a method of killing or inhibiting the growth of a fungus, the method includes contacting the fungus with the composition of the present invention, in an amount and for a period of time effective to kill or inhibit the growth of the fungus.

The present invention also provides a method of killing or inhibiting the growth of a virus, the method includes contacting the virus with the composition of the present invention, in an amount and for a period of time effective to kill or inhibit the growth of the virus.

The present invention also provides a method of killing or inhibiting the growth of a bacteria, the method includes contacting the bacteria with the composition of the present invention, in an amount and for a period of time effective to kill or inhibit the growth of the bacteria.

The present invention also provides a method of treating a disorder in a mammal, the method includes contacting the mammal with the composition of the present invention, in an amount and for a period of time effective to treat the disorder.

The present invention also provides a method of treating a disorder that is associated with unplugging of tympanostomy tubes; tinnitus; inner ear toxicity; hearing loss; vestibular disorders; facial nerve palsy; cerumen impaction; prevention of tympanic membrane scarring secondary to infection or pressure equalization tubes; improvement of Eustachian tube function; cholesterol granuloma; modulation of fibroblast or keratinocyte activity; dissolution of cholesteatoma or other keratotic debris; reduction of granulation tissue; dissolution of surgical packing material; and combinations thereof.

The present invention also provides a method for preserving contact lens, the method includes contacting the contact lens with the composition of the present invention, in an amount and for a period of time effective to preserve the contact lens.

The present invention also provides a method for cleansing surgical or dental instrument, the method includes contacting the surgical or dental instrument with the composition of the present invention, in an amount and for a period of time effective to cleanse surgical or dental instrument.

The present invention also provides a kit that includes: (a) a container that includes the pharmaceutical composition of the present invention; and (b) a drug delivery device.

The present invention also provides a kit wherein the drug delivery device is an eye dropper, an ear dropper, a pressure equalization tube, a pledget, an osmotic device, a micropump, a micro-wick, a prosthetic device, iontophoresis, powder, a cochlear implant, an ossicular prostheses or bone cement.

The kit of the present invention may also provide a packaging material in which the container and drug delivery device are stored.

The kit of the present invention may also provide a printed indicia or instructions located on the packaging material.

The present invention also provides a kit wherein the packaging material is air tight (configured to exclude oxygen).

The present invention also provides a kit wherein the container is a disposable ampule.

The present invention also provides a kit wherein the disposable ampule is a single dose ampule.

The present invention also provides a kit wherein the container is air tight (configured to exclude oxygen).

### DETAILED DESCRIPTION OF THE INVENTION

Reference will now be made in detail to certain claims of the invention, examples of which are illustrated in the accompanying structures and formulas. While the invention will be described in conjunction with the enumerated claims, it will be understood that they are not intended to limit the invention to those claims. On the contrary, the invention is intended to cover all alternatives, modifications, and equivalents, which may be included within the scope of the present invention as defined by the claims.

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The present invention relates to pharmaceutical compositions, method of killing or inhibiting the growth of a fungus, methods of killing or inhibiting the growth of a virus, methods of killing or inhibiting the growth of a bacteria, methods of treating a disorder in a mammal, methods for preserving contact lens, methods for cleansing surgical or dental instrument, and kits. When describing the pharmaceutical compositions, method of killing or inhibiting the growth of a fungus, methods of killing or inhibiting the growth of a virus, methods of killing or inhibiting the growth of a bacteria, methods of treating a disorder in a mammal, methods for preserving contact lens, methods for cleansing surgical or dental instrument, and kits, the following terms have the following meanings, unless otherwise indicated.

### DEFINITIONS

Unless stated otherwise, the following terms and phrases as used herein are intended to have the following meanings:

When tradenames are used herein, applicants intend to independently include the tradename product and the active pharmaceutical ingredient(s) of the tradename product.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

As used herein, “pharmaceutically acceptable salts” refer to compounds described herein, wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the compounds described herein can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., (1985), 1418 the disclosure of which is hereby incorporated by reference.

“Therapeutically effective amount” is intended to include an amount of a compound described herein, or an amount of the combination of compounds described herein, e.g., to treat or prevent the disease or disorder, or to treat the symptoms of the disease or disorder, in a host. The combination of compounds is preferably a synergistic combination. Synergy, as described for example by Chou and Talalay, Adv. Enzyme Regul., 22:27 (1984), occurs when the effect of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

As used herein, “treating” or “treat” includes (i) preventing a pathologic condition from occurring (e.g. prophylaxis); (ii) inhibiting the pathologic condition or arresting its development; (iii) relieving the pathologic condition; and/or (iv) diminishing symptoms associated with the pathologic condition.

The compounds described herein can be administered as the parent compound, a pro-drug of the parent compound, or an active metabolite of the parent compound.

“Metabolite” refers to any substance resulting from biochemical processes by which living cells interact with the active parent drug or other formulas or compounds of the present invention in vivo, when such active parent drug or other formulas or compounds of the present are administered to a mammalian subject. Metabolites include products or intermediates from any metabolic pathway.

“Metabolic pathway” refers to a sequence of enzyme-mediated reactions that transform one compound to another and provide intermediates and energy for cellular functions. The metabolic pathway can be linear or cyclic.

“Pro-drugs” are intended to include any covalently bonded substances which release the active parent drug or other formulas or compounds of the present invention in vivo when such pro-drug is administered to a mammalian subject. Pro-drugs of a compound of the present invention are prepared by modifying functional groups present in the compound in such a way that the modifications are cleaved, either in routine manipulation in vivo, to the parent compound. Pro-drugs include compounds of the present invention wherein the carbonyl, carboxylic acid, hydroxy or amino group is bonded to any group that, when the pro-drug is administered to a mammalian subject, cleaves to form a free carbonyl, carboxylic acid, hydroxy or amino group. Examples of pro-drugs include, but are not limited to, acetate, formate and benzoate derivatives of alcohol and amine functional groups in the compounds of the present invention, and the like.

Pro-drugs include hydroxyl and amino derivatives well-known to practitioners of the art, such as, for example, esters prepared by reaction of the parent hydroxyl compound with a suitable carboxylic acid, or amides prepared by reaction of the parent amino compound with a suitable carboxylic acid. Simple aliphatic or aromatic esters derived from hydroxyl groups pendent on the compounds employed in this invention are preferred pro-drugs. In some cases it may be desirable to prepare double ester type pro-drugs such as (acyloxy) alkyl esters or ((alkoxycarbonyl)oxy)alkyl esters. Specific suitable esters as pro-drugs include methyl, ethyl, propyl, isopropyl, n-butyl, isobutyl, tert-butyl, and morpholinoethyl.

### Antibiotic

The composition of the present invention can further include an antibiotic. As used herein, an “antibiotic” is any compound having activity against either Gram-positive or Gram-negative organisms (i.e., inhibits the growth or destroys the development of either Gram-positive or Gram-negative organisms). Stedman's Medical Dictionary, Illustrated, (25th Ed.), Williams & Wilkins: Baltimore (1990) and Mosby's Medical, Nursing, & Allied Health Dictionary, (5th Ed.), Mosby: St. Louis (1998).

Suitable antibiotics are disclosed, e.g., in Physician's Desk Reference (PDR), Medical Economics Company (Montvale, N.J.), (53rd Ed.), 1999; Mayo Medical Center Formulary, Unabridged Version, Mayo Clinic (Rochester, Minn.), January 1998; Merck Index, An Encyclopedia of Chemicals, Drugs, and Biologicals, (11th Ed.), Merck & Co., Inc. (Rahway, N.J.), 1989; University of Wisconsin Antimicrobial Use Guide, http://www.medsch.wisc.edu/clinsci/amcg/amcg.html; Introduction on the Use of the Antibiotics Guideline, Descriptions of Specific Antibiotic Classes, Thomas Jefferson University, http://jeffline.tju.edu/CWIS/OAC/antibiotics_guide/intro.html; and references cited therein.

Suitable antibiotics include, e.g., β-lactam antibiotics, cephalosporins, macrolides, miscellaneous antibiotics, penicillins, tetracyclines, antifungals, antimalarial agents, antituberculosis agents, antivirals, leprostatics, miscellaneous anti-infectives, quinolones, sulfonamides, urinary anti-infectives, nasal antibiotics, ophthalmic antibiotics, ophthalmic antivirals, ophthalmic quinolones, ophthalmic sulfonamides, skin and mucous membrane antibiotics, skin and mucous membrane antifungals, skin and mucous membrane antivirals, skin and mucous membrane miscellaneous anti-infectives, skin and mucous membrane scabicides and pediculicides, skin and mucous membrane antineoplasts, nitrofurans, and oxazolidinones. Physician's Desk Reference (PDR), Medical Economics Company (Montvale, N.J.), (53rd Ed.), 1999 and Mayo Medical Center Formulary, Unabridged Version, Mayo Clinic (Rochester, Minn.), January 1998.

β-Lactam antibiotics include, e.g., Azactam (aztreonam); Lorabid (loracarbef); Merrem (meropenem); and Primaxin (imipenem and cilastatin for injectable suspension).

Cephalosporins include, e.g., Ancef (cefazolin); Ceclor (cefaclor); Cefotan (cefotetan); Cedax (ceftibuten); Cefizox (ceftizoxime sodium); Cefobid (cefoperazone sodium); Ceftin (cefuroxime axetil); Cefzil (cefprozil); Ceptaz (ceftazidime); Claforan (cefotaxime); Duricef (cefadroxil monohydrate); Fortaz (ceftazidime); Keflex (cephalexin); Keftab (cephalexin HCl); Kefurox (cefuroxime); Kefzol (cefazolin); Mandol (cefamandole nafate); Maxipime (cefepime HCl); Mefoxin (cefoxitin); Monocid (cefonicid sodium); Omnicef (cefdinir); Rocephin (ceftriaxone); Suprax (cefixime); Tazicef (ceftazidime); Tazidime (ceftazidime); Vantin (cefpodoxime proxetil); and Zinacef (cefuroxime).

Macrolides include, e.g., Biaxin (clarithromycin); Dynabac (dirithromycin); E.E.S. 200 (Erythromycin Ethylsuccinate); E.E.S. 400 (Erythromycin Ethylsuccinate); Ery-Ped 200 (Erythromycin Ethylsuccinate); EryPed 400 (Erythromycin Ethylsuccinate); Ery-Tab (Erythromycin delayed-release tablets); Erythrocin Stearate (Erythromycin stearate); Ilosone (erythromycin estolate); PCE Dispertab (erythromycin particles in tablets); Pediazole (erythromycin ethylsuccinate and sulfisoxazole acetyl for oral suspension); Tao (troleandomycin); Zithromax (azithromycin); and Erythromycin.

Miscellaneous antibiotics include, e.g., Cleocin HCl (clindamycin hydrochloride); Cleocin Phosphate (clindamycin phosphate); Coly-Mycin M (colistimethate sodium); and Vancocin HCl (vancomycin hydrochloride).

Penicillins include, e.g., Amoxil (amoxicillin); Augmentin (amoxicillin/clavulanate potassium); Bicillin C-R 900/300 (Penicillin G benzathine and Penicillin G procaine suspension); Bicillin C-R (Penicillin G benzathine and Penicillin G procaine suspension); Bicillin L-A (Penicillin G benzathine suspension); Geocillin (carbencillin indanyl sodium); Mezlin (sterile mezlocillin sodium); Omnipen (ampicillin); Pen-Vee K (penicillin V potassium); Pfizerpen (penicillin G potassium); Pipracil (piperacillin sodium); Spectrobid (bacampicillin HCl); Ticar (ticarcillin disodium); Timentin (ticarcillin disodium and clavulanate potassium); Unasyn (ampicillin sodium/sulbactam sodium); Zosyn (piperacillin sodium and tazobactam sodium); and Dicloxacillin Sodium.

Tetracyclines include, e.g., Achromycin V (tetracycline HCl); Declomycin (demeclocycline HCl); Dynacin (minocylcine HCl); Minocin (minocycline hydrochloride); Monodox (Doxycycline monohydrate capsules); Terramycin (oxytetracyline); Vectrin (minocycline hydrochloride); Vibramycin Calcium (doxycycline sodium); Vibramycin Hyclate (doxycycline hyclate); Vibramycin Monohydrate (doxycycline monohydrate); Vibra-Tabs (doxycycline hydrate); Declomycin (demeclocycline HCl); Vibramycin (doxycycline); Dynacin (Minocyline HCl); Terramycin (oxytetracycline HCl); Achromycin V capsules (tetracycline HCl); Lincomycins; and Cleocin HCl (clindamycin HCl).

Antifungals include, e.g., Abelcet (amphotericin B lipid complex); AmBisome (amphotericin B); Amphotec (amphotericin B cholesterol sulfate complex); Ancobon (flucytosine); Diflucan (fluconazole); Fulvicin P/G (ultramicrosize griseofulvin); Fulvicin P/G 165 and 330 (ultramicrosize griseofulvin); Grifulvin V (griseofulvin); Gris-PEG (griseofulvin ultramicrosize); Lamisil (terbinafine hydrochloride); Nizoral (ketoconazole); Amphotericin B; Lotrimin (clotrimazole); Dapsone tablets (dapsone); Diflucan (fluconazole); Monistat-Derm cream (miconazole); Mycostatin Cream (nystatin); and Sporanox (itraconazole).

Antimalarial agents include, e.g., Aralen hydrochloride (chloroquine HCl); Aralen phosphate (chloroquine phosphate); Daraprim (pyrimethamine); Lariam (mefloquine HCl); and Plaquenil (hydroxychloroquine sulfate).

Antituberculosis agents include, e.g., Capastat sulfate (capreomycin sulfate); Myambutol (ethambutol hydrochloride); Mycobutin (rifabutin capsules); Nydrazid (isoniazid injection); Paser (aminosalicylic acid); Priftin (rifapentine); Pyrazinamide tablets (pyrazinamide); Rifadin (rifampin capsules); Rifadin IV (rifampin for injection); Rifamate (rifampin and isoniazid); Rifater (rifampin, isoniazid and pyrazinamide); Seromycin (cycloserine capsules); Streptomycin Sulfate; Tice BCG (BCG vaccine); Cycloserine (seromycin capsules); Urised (Methenamine); and Trecator-SC (ethionamide tablets).

Antivirals include, e.g., Alferon N (interferon alfa-n3); Crixivan (indinavir sulfate); Cytovene (ganciclovir); Cytovene-IV (ganciclovir sodium); Epivir (lamivudine); Famvir (famciclovir); Flumadine (rimantadine HCl); Foscavir (foscamet sodium); Hivid (zalcitabine); Intron A (interferon alfa-2b); Invirase (saquinavir mesylate); Norvir (ritonavir); Rebetron combination therapy, which contains Rebetrol (ribavirin) and Intron A (inteferon alfa-2b); Rescriptor (delavirdine mesylate); Retrovir (ziduvudine); Retrovir IV (zidovudine); Symmetrel (amantadine hydrochloride); Synagis (palivizumab); Valtrex (valacyclovir HCl); Videx (didanosine); Viracept (nelfinavir mesylate); Viramune (nevirapine); Virazole (ribavirin); Vistide (cidofovir); Zerit (stavudine (d4T)); Symmetrel Syrup (amantadine HCl); Combivir Tablets (lamiduvine); and Zovirax (acyclovir).

Leprostatics include, e.g., Dapsone Tablets (dapsone).

Miscellaneous anti-infectives include, e.g., Daraprim (pyrimethamine); Flagyl 375 (metronidazole); Flagyl ER Tablets (metronidazole); Flagyl I.V. (metronidazole); Furoxone (furazolidone); Mepron (atovaquone); and Neutrexin (trimetrexate glucuronate).

Quinolones include, e.g., Cipro (ciprofloxacin HCl); Floxin (ofloxacin); Levaquin (levofloxacin); Mazaquin (lomefloxacin HCl); Noroxin (norfloxacin); Penetrex (enoxacin); Raxar (grepafloxacin HCl); Trovan (trovafloxacin mesylate); and Zagam (sparfloxacin).

Sulfonamides include, e.g., Bactrim (trimethoprim and sulfamethoxazole); Bactrim DS (trimethoprim and sulfamethoxazole double strength); Pediazole (erythromycin ethylsuccinate and sulfisaxazole acetyl); Septra (trimethoprim and sulfamethoxazole); Septra DS (trimethoprim and sulfamethoxazole); Co-Trimoxazole, Sulfadiazine, Bactrim I.V. Infusion (sulfamethoxazole); Sulfapyridine, and Pediazole (erythromycin ethylsuccinate and sulfisoxazole acetyl).

Urinary anti-infectives include, e.g., Furadantin (nitrofurantoin); Macrobid (nitrofurantoin monohydrate macrocrystals); Macrodantin (nitrofurantoin macrocrystals); Monurol Sachet (fosfomycin tromethamine); NegGram Caplets (nalidixic acid); Septra (trimethoprim and sulfamethoxazole); Septra DS (trimethoprim and sulfamethoxazole); Urised (a combination of the antiseptics methenamine, methylene blue, phenyl salicylate, benzoic acid and parasympatholytics (atropine sulfate) hyoscyamine); Urobiotic-250 Capsules (oxytetracycline HCl, sulfamethizole and phenazopyridine HCl); and Uroqid Acid No. 2 Tablets (methenamine mandelate).

Nasal antibiotics include, e.g., Bactroban (mupirocin).

Ophthalmic antibiotics include, e.g., Chloromycetin ophthalmic (chloramphenical); Cortisporin (neomycin and polymyxin β sulfates and hydrocortisone acetate cream); Ilotycin (erythromycin ophthalmic ointment); NeoDecadron (neomycin sulfate-dexamethasone sodium phosphate); Polytrim (trimethoprim and polythyxin, sulfate ophthalmic solution); Terra-Cortril (oxytetracycline HCl and hydrocortisone acetate); Terramycin (oxytetracycline); and TobraDex (tobramycin and dexamethasone ophthalmic suspension and ointment).

Ophthalmic antivirals include, e.g., Vira-A ophthalmic ointment, (vidarabine).

Ophthalmic quinolones include, e.g., Chibroxin (norfloxacin ophthalmic solution); Ciloxan ophthalmic solution, (Ciprofloxacin HCl); Ciloxan ophthalmic ointment, (Ciprofloxacin HCl); and Ocuflox ophthalmic solution (ofloxacin).

Ophthalmic sulfonamides include, e.g., Blephamide ophthalmic ointment (sulfacetamide sodium and prednisolone acetate); and Blephamide ophthalmic suspension (sulfacetamide sodium and prednisolone acetate).

Skin and mucous membrane antibiotics include, e.g., A/T/S (erythromycin); Bactroban (mupirocin); Benzamycin (erythromycin-benzoyl peroxide topical gel); Betadine (povidone-iodine); Cleocin T (clindamycin phosphate topical solution); Clindets (clindamycin phosphate pledgets); Cortisporin (neomycin, polymyxin B sulfates and hydrocortisone acetate cream); Emgel (erythromycin); Erycette (erythromycin topical solution); Garamycin (gentamicin sulfate); Klaron (sodium sulfacetamide lotion); Mycostatin (nystatin cream); Theramycin Z (erythromycin topical solution); T-Stat (erythromycin); Chloromycetin (chloramphenicol ophthalmic ointment); Cortisporin (neomycin and polymyxin B sulfates, bacitracin zinc and hydrocortisone ophthalmic ointment); Ilotycin (erythromycin); NeoDecadron (neomycin sulfate-dexamethasone sodium phosphate); Polytrim (trimethoprim and polymyxin B sulfate); Terra-Cortril (oxytetracycline HCl and hydrocortisone acetate); Terramycin (oxytetracycline); and TobraDex (tobramycin and dexamethasone ophthalmic suspension and ointment).

Skin and mucous membrane antifungals include, e.g., Exelderm (sulconazole nitrate); Fungizone (amphotericin B oral suspension); Larnisil (terbinafine hydrochloride cream); Loprox (ciclopiroxolamine); Lotrimin (clotrimazole); Lotrisone (clotrimazole and betamethasone diproprionate); Mentax (butenafine HCl); Monistat-Derm (miconazole nitrate); Mycelex (clotrimazole); Mycostatin (nystatin); Naftin (naftifine HCl); Nizoral (ketoconazole); Nystop (nystatin); Oxistat (oxiconazole nitrate); Selsun Rx (2.5% selenium sulfide lotion); and Spectazole (econazole nitrate).

Skin and mucous membrane antivirals include, e.g., Denavir (penciclovir cream); and Zovirax (acyclovir).

Skin and mucous membrane miscellaneous anti-infectives include, e.g., Benzashave (benzoyl peroxide); Betadine (povidone-iodine); Betasept (chlorhexidine gluconate); Cetaphil (soap substitute); Clorpactin WCS-90 (sodium oxychlorosene); Dapsone Tablets (dapsone); Desquam-E (benzoyl peroxide); Desquam-X (benzoyl peroxide); Hibiclens (chlorhexidine gluconate); Hibistat (chlorhexidine gluconate); Impregon (tetrachlorosalicylanilide 2%); MetroCream (metronidazole); MetroGel (metronidazole); Noritate (metronidazole); pHisoHex (hexachlorophene detergent cleanser); Sulfacet-R (sodium sulfacetamide 10% and sulfur 5%); Sulfamylon (matenide acetate); Triaz (benzoyl peroxide); and Vanoxide-HC (benzoyl peroxide hydrocortisone).

Skin and mucous membrane scabicides and pediculicides include, e.g., Acticin (permethrin); Elimite (permethrin); Eurax (crotamiton); and Lindane Lotion USP 1% (lindane).

Skin and mucous membrane antineoplasts include, e.g., Efudex (fluorouracil); and Fluoroplex (fluorouracil).

Nitrofurans include, e.g., Furadantin Oral Suspension (nitrofurantoin).

Oxazolidinones include, e.g., Zyvox (linezolid).

It is appreciated that those skilled in the art understand that the antibiotic useful in the present invention is the biologically active compound present in any of the antibiotic formulations disclosed above. For example, Azactam (aztreonam) is typically available as an injectable solution. The antibiotic, however, is (z)-2-[[[(2-amino-4-thiazolyl) [[(2S,-3S)-2-methyl-4-oxo-1-sulfo-3-azetidinyl]carbamoyl]methylene]amino]oxy]-2-methyl propionic acid. Physician's Desk Reference (PDR), Medical Economics Company (Montvale, N.J.), (53rd Ed.), pp. 820-823, 1999.

Azactam (aztreonam) is commercially available from Bristol-Myers Squibb and is (Z)-2-[[[(2-amino-4-thiazolyl) [[(2S,-3S)-2-methyl-4-oxo-1-sulfo-3-azetidinyl]carbamoyl]methylene]amino]oxy]-2-methylpropionic acid.

Cefotan (cefotetan) is commercially available from Zeneca and is the disodium salt of [6R-(6a, 7a)]-7-[[[4-(2-amino-1-carboxy-2-oxoethylidene)-1-3, dithietan-2-yl]carbonyl]amino]-7-methoxy-3-[[(1-methyl-1H-tetrazol-5-yl) thio]methyl]-8-oxo-5-thia-1-azabicyclo[4.2.0]oct-2-ene-2-carboxylic acid.

Lorabid (loracarbef) is commercially available from Lilly and is (6R,7S)-7-[(R)-2-amino-2-phenylacetamido]-3-chloro-8-oxo-1-azabicyclo[4.2.0]oct-2-ene-2-carboxylic acid, monohydrate.

Mefoxin (cefoxitin) is commercially available from Merck and is sodium (6R,7S)-3-(hydroxymethyl)-7-methoxy-8-oxo-7-[2-(2-thienyl)acetamido]-5-thia-1-azabicylo[4.2.0]oct-2-ene-2-carboxylate carbamate (ester).

Merrem (meropenem) is commercially available from Zeneca and is (4R,5S, 6S)-3-[(3S,5S)-5-(Dimethylcarbamoyl)-3-pyrrolidinyl]thiol]-6-[(1R)-1-hydroxyethyl]-4-methyl-7-oxo-1-azabicyclo[3.2.0]hept-2-ene-2-carboxylic acid trihydrate.

Primaxin (imipenem and cilastatin for injectable suspension) is commercially available from Merck and is (1) imipenem is N-formimidoylthienamycin monohydrate, chemical name is [5R-[5α,6α(R*)]]-6-(1-hydroxyethyl)-3-[[2-[(iminomethyl)amino]ethyl]thio]-7-oxo-1-azabicylco[3.2.0]hept-2-ene-2-carboxylic acid monohydrate, cilastatin sodium is [R—[R*, S*,-(Z)]]-7-[(2-amino-2-carboxyethyl) thio]-2-[[(2,2-dimethyl cyclopropyl) carbonyl]amino]-2-heptenoic acid, monosodium salt.

Ancef (cefazolin) is commercially available from SmithKline Beecham and is 3-{[(5-methyl-1,3,4-thiadiazol-2-yl) thio-methyl]}-8-oxo-7-[2-(1H-tetrazol-1-yl)acetamido]-5-thia-1-azabicyclo[4.2.0]oct-2-ene-2-carboxylic acid.

Ceclor (cefaclor) is commercially available from Lilly and is 3-chloro-7-D-(2-phenylglycinamido)-3-cephem-4-carboxylic acid monohydrate; Cedax (ceftibuten) is commercially available from Schering and is (+)-(6R,7R)-7-[(Z)-2-(2-(2-amino-4-thiazoly)-4-carboxycrotonamido]-8-oxo-5-thia-1-azabicyclo[4.2.0]oct-2-ene-2-carboxylic acid, dihydrate.

Cefizox (ceftizoxime sodium) is commercially available from Fujisawa and is sodium salt of [6R-[6α,7β(Z)]]-7 [[2,3, dihydro-2-imino-4-thiazolyl)(methoxy amino)acetyl]amino]-8-oxo-5-thia-1-azabicyclo[4.2.0]oct-2-ene-2-carboxyolic acid.

Cefobid (cefoperazone sodium) is commercially available from Pfizer and is sodium (6R,7R)-7[(R)-2-(4-ethyl-2,3, dioxo-1-piperazine carboxamido)-2-(p-hydroxyphenyl)-acetamido)-3-[[(1-methyl-1H-tetrazol-5-yl) thio]methyl]-8-oxo-5-thia-1-azabicyclo[4.2.0]oct-2-ene-2-carboxylate.

Ceftin (cefuroxime axetil) is commercially available from Glaxo Wellcome and is (RS)-1-hydroxyethyl (6R,7R)-7-[2-(2-furyl) glyoxylamido]-3-(hydroxyethyl)-(8-oxo-5-thia-1-aza-bicyclo[4.2.0]oct-2-ene-2-carboxylate, 72 (Z) —O-methyl-oxime), 1-acetate 3-carbamate.

Cefzil (cefprozil) is commercially available from Bristol-Myers Squibb and is (6R,7R)-7-[(R)-2-amino-2-(p-hydroxyphenyl)acetamido]-8-oxo-3-propenyl-5-thia-1-azabicyclo[4.2.0]oct-2-ene-2-carboxylic acid monohydrate.

Ceptaz (ceftazidime) is commercially available from Glaxo Wellcome and is 1-[[7-[[(2-amino-4-thiazolyl) [(1-carboxy-1-methylethoxy) imine]acetyl]amino]-2-carboxy-8-oxo-5-thia-1-azabicyclo[4.2.0]oct-2-en-3-yl]methyl]-, hydroxide, inner salt, [6R-[6α,7β(Z)]].

Claforan (cefotaxime) is commercially available from Hoescht Marion Roussel and is 7-[2-(2-amino-4-thiazolyl) glyoxylamido]-3-(hydroxy methyl)-8-oxo-5-thia-1-azabicyclo[4.2.0]oct-2-ene-2-carboxylate 72 (Z)-(O-methyloxime), acetate (ester).

Duricef (cefadroxil monohydrate) is commercially available from Bristol-Myers Squibb and is 5-Thia-1-azabicyclo[4.2.0]oct-2-ene-2-carboxylic acid, 7-[[amino (4-hydroxyphenyl)acetyl]amino]-3-methyl-8-oxo,-monohydrate, [6R-[6α, 7β(R*)]]-.

Fortaz (ceftazidime) is commercially available from Glaxo Wellcome and is 1-[[7-[[(2-amino-4-thiazolyl) [1-carboxy-1-methylethoxy) imino]acetyl]amino]-2-carboxy-8-oxo-5-thia-1-azabicyclo[4.2.0]oct-2-en-3-yl]methyl]-, hydroxide, inner salt [6R-[6α,7β(Z)]].

Keflex (cephalexin) is commercially available from Dista and is 7-(D-α-Amino-α-phenyl acetamido)-3-methyl-3-cephem-4-carboxylic acid monohydrate.

Keftab (cephalexin HCl) is commercially available from Dura and is 7-(D-2-Amino-2-phenylacetamido)-3-methyl-3-cephem-4-carboxylic acid hydrochloride monohydrate.

Kefurox (cefuroxime) is commercially available from Lilly and is the sodium salt of (6R,7R) 3-carbamoyloxymethyl-7-[Z-2-methoxyimino-2-(fur-2-yl)acetamiodo]ceph-3-em-4-carboxylate.

Kefzol (cefazolin) is commercially available from Lilly and is the sodium salt of 3-{[(5-methyl-1,3,4-thiadiazol-2-yl)thio]methyl}-8-oxo-7-[2-(1H-tetrazol-1-yl)acetamido]-5-thia-1-azabicyclo[4.2.0]oct-2-ene-2-carboxylic acid.

Mandol (cefamandole nafate) is commercially available from Lilly and is 5-thia-1-azabicyclo[4.2.0]oct-2-ene-2-carboxylic acid, 7-[[(formyloxy)phenyl acetyl]amino]-3-[[(1-methyl-1H-tetrazol-5-yl)thio]methyl]-8-oxo-, mono-sodium salt, [6R-[6α-7β(R*)]].

Maxipime (cefepime HCl) is commercially available from Bristol-Myers Squibb and is 1-[[6R, 7R)-7-[2-amino-4-thiazolyl)-glyoxylamido]-2-carboxy-8-oxo-5-thia-1-azabicyclo-[4.2.0]oct-2-en-3-yl]methyl]-1-methylpyrrolidinium chloride, 72-(Z)-(O-methyloxime), monohydrochloride, monohyrate.

Monocid (cefonicid sodium) is commercially available from SmithKline Beecham and is 5-Thia-1-azabicyclo[4.2.0]oct-2-ene-2-carboxylic acid, 7-[(hydroxyphenyl-acetyl)-amino]-8-oxo-3-[[1-(sulfomethyl)-1H-tetrazol-5-yl]thio]methyl]-disodium salt, [6R-[6α,7β(R*)]].

Omnicef (cefdinir) is commercially available from Parke Davis and is [6R-[6α,7β(Z)]]-7-[[(2-amino-4-thiazolyl)-(hydroxyimino) acetyl]amino]-3-ethenyl-8-oxo-5-thia-1-azabicyclo[4.2.0]oct-2-ene-2-carboxylic acid.

Rocephin (ceftriaxone) is commercially available from Roche Laboratories and is (6R,7R)-7-[2-(2-Amino-4-thiazolyl) glyoxylamido]-8-oxo-3-[[(1,2,5,6-tetrahydro-2-methyl-5,6-dioxo-as-triazin-3-yl) thio]methyl]-5-thia-1-azabicyclo [4.2.0]oct-2-ene-2-carboxylic acid, 72-(Z)-O-methyloxime), disodium salt, sesquaterhydrate.

Suprax (cefixime) is commercially available from Lederle Laboratories and is (6R,7R)-7-[2-(2-Amino-4-thiazolyl) glyoxylamido]-8-oxo-3-vinyl-5-thia-1-azabicyclo[4.2.0]oct-2-ene-2-carboxylic acid, 72-(Z)-[O-(carboxymethyl) oxime]trihydrate.

Tazicef (ceftazidime) is commercially available from SmithKline Beecham and is pentahydrate of Pyridinium, 1-[[7-[[2-amino-4-thiazolyl) [(1-carboxy-1-methylethoxy) imino]acetyl]amino]-2-carboxy-8-oxo-5-thia-1-azabicyclo (4.2.0) oct-2-en-3-yl]methyl]-hydroxide, inner salt, [6R,-[6α,7β(Z)]].

Tazidime (ceftazidime) is commercially available from Lilly and is pentahydrate of Pyridinium, 1-[[7-[[2-amino-4-thiazolyl) [(1-carboxy-1-methylethoxy) imino]acetyl]amino]-2-corboxy-8-oxo-5-thia-1-azabicyclo (4.2.0) oct-2-en-3-yl]methyl]-hydroxide, inner salt, [6R,-[6α,7β(Z)]].

Vantin (cefpodoxime proxetil) is commercially available from Pharmacia & Upjohn and is (RS)-1 (isopropoxycarbonyloxy)ethyl (+)-(6R,7R)-7-[2-(2-amino-4-thiazolyl)-2-{(Z)methoxyimino}acetamido]-3-methoxymethyl-8-oxo-5-thia-1-azabicyclo[4.2.0]oct-2-ene-2-carboxylate.

Zinacef (cefuroxime) is commercially available from Glaxo Wellcome and is sodium salt of (6R,7R)-3-carbamoyloxymethyl-7-[Z-2-methoxy-imino-2-fur-2-yl)acetamido]ceph-3-em-4-carboxylate.

Biaxin (clarithromycin) is commercially available from Abbott and is 6-O-methylerythromycin.

Dynabac (dirithromycin) is commercially available from Sanofi and is (9S)-9-Deox-11-deoxy-9,11-[imino[(1R)-2-(2-methoxyethoxy)-ethylidene]oxy]erythromycin.

E.E.S. 200 (Erythromycin Ethylsuccinate) is commercially available from Abbott and is erythromycin 2′-(ethylsuccinate).

E.E.S. 400 (Erythromycin Ethylsuccinate) is commercially available from Abbott and is erythromycin 2′-(ethylsuccinate).

Ery-Ped 200 (Erythromycin Ethylsuccinate) is commercially available from Abbott and is erythromycin 2′-(ethylsuccinate).

EryPed 400 (Erythromycin Ethylsuccinate) is commercially available from Abbott and is erythromycin 2′-(ethylsuccinate).

Ery-Tab (Erythromycin delayed-release tablets) is commercially available from Abbott and is (3R*, 4S*, 5S*, 6R*, 7R*, 9R*, 11R*, 12R*, 13S*, 14R*)-4-[(2,6-dideoxy-3-C-methyl-3-O-methyl-α-L-ribo-hexopyranosyl) oxy]-14-ethyl-7, 12,13-trihydroxy-3,5,7,9,11,13, hexamethyl-6-[[3,4,6-trideoxy-3-(dimethylamino)-β-D-xylo-hex-opyranosyl]oxy]oxacyclotetradecane-2,10-dione.

Erythrocin Stearate (Erythromycin stearate) is commercially available from Abbott and is the stearate salt of (3R*, 4S*, 5S*, 6R*, 7R*, 9R*, 11R*, 12R*, 13S*, 14R*)-4-[(2,6-dideoxy-3-C-methyl-3-O-methyl-α-L-ribo-hexopyranosyl) oxy]-14-ethyl-7,12,13-trihydroxy-3,5,7,9,11,13, hexamethyl-6-[[3,4,6-trideoxy-3-(dimethylamino)-β-D-xylo-hex-opyranosyl]oxy]oxacyclotetradecane-2,10-dione.

Ilosone (erythromycin estolate) is commercially available from Dista and is erythromycin 2′-propionate, dodecyl sulfate.

PCE Dispertab (erythromycin particles in tablets) is commercially available from Abbott and is (3R*, 4S*, 5S*, 6R*, 7R*, 9R*, 11R*, 12R*, 13S*, 14R*)-4-[(2,6-dideoxy-3-C-methyl-3-O-methyl-α-L-ribo-hexopyranosyl) oxy]-14-ethyl-7, 12,13-trihydroxy-3,5,7,9,11,13, hexamethyl-6-[[3,4,6-trideoxy-3-(dimethylamino)-β-D-xylo-hex-opyranosyl]oxy]oxacyclotetradecane-2,10-dione.

Pediazole (erythromycin ethylsuccinate and sulfisoxazole acetyl for oral suspension) is commercially available from Ross Products and is 2′-ethylsuccinyl ester of erythromycin (erythromycin ethylsuccinate) and N-(3,4-dimethyl-5-isoxazolyl)-N-sulfanilylacetamide (sulfisoxazole acetyl).

Tao (troleandomycin) is commercially available from Pfizer and is the synthetically derived acetylated ester of oleandomycin.

Zithromax (azithromycin) is commercially available from Pfizer and is (2R, 3S, 4R, 5R, 8R, 10R, 11R, 12S, 13S, 14R)-13-[(2,6-dideoxy-3-C-methyl-3-O-methyl-α-L-ribo-hexopyranosyl) oxy]-2-ethyl-3,4,10-trihydroxy-3,5,6,8,10,12, 14-heptamethyl-11-[[3,4,6-trideoxy-3-(dimethylamino)-β-D-xylo-hexopyranosyl]oxy]-1-oxa-6-azacyclopentadecan-15-one.

Erythromycin, which is (3R*, 4S*, 5S*, 6R*, 7R*, 9R*, 11R*, 12R*, 13S*, 14R*)-4-[(2,6-dideoxy-3-C-methyl-3-O-methyl-α-L-ribo-hexopyranosyl) oxy]-14-ethyl-7,12,13-trihydroxy-3,5,7,9,11,13, hexamethyl-6-[[3,4,6-trideoxy-3-(dimethylamino)-11-D-xylo-hex-opyranosyl]oxy]oxacyclotetradecane-2,10-dione.

Cleocin HCl (clindamycin hydrochloride) is commercially available from Pharmacia & Upjohn and is the hydrated hydrochloride salt of clindamycin, a semisynthetic antibiotic produced by a 7 (S)-chlorosubstitution of the (7R) hydroxyl group of lincomycin.

Cleocin Phosphate (clindamycin phosphate) is commercially available from Pharmacia & Upjohn and is L-threo-α-D-galacto-Octopyranoside, methyl 7 chloro-6,7,8,-trideoxy-6-[[(1-methyl-4-propyl-2-pyrrolidinyl) carbonyl]amino]-1-thio-, 2-(dihydrogen phosphate), (2S-trans)-.

Coly-Mycin M (colistimethate sodium) is commercially available from Monarch.

Vancocin HCl (vancomycin hydrochloride) is commercially available from Lilly.

Amoxil (amoxicillin) is commercially available from SmithKline Beecham and is (2S,5R,6R)-6-[(R)-(−)-2-amino-2-(p-hydroxyphenyl)acetamido]-3,3-dimethyl-7-oxo-4-thia-1-azabicyclo[3.2.0]-heptane-2-carboxylic acid trihydrate.

Augmentin (amoxicillin/clavulanate potassium) is commercially available from SmithKline Beecham and is (2S,5R,6R)-6-[(R)-(−)-2-amino-2-(p-hydroxy phenyl)acetamido]-3,3-dimethyl-7-oxo-4-thia-1-azabicyclo[3.2.0]-heptane-2-carboxylic acid trihydrate (amoxicillin) and potassium (Z)-(2R,5R)-3-(2-hydroxyethylidene)-7-oxo-4-oxa-1-azabicyclo[3.2.0]-heptane-2-carboxylate (clavulanate potassium).

Bicillin C—R 900/300 (Penicillin G benzathine and Penicillin G procaine suspension) is commercially available from Wyeth-Ayerst and is (2S,5R,6R)-3,3-Dimethyl-7-oxo-6-(2-phenylacetamido)-4-thia-1-azabicyclo[3.2.0]heptane-2-carboxylic acid compound with N,N′-dibenzylethylenediamine (2:1), tetrahydrate (Penicillin G benzathine) and (2S,5R,6R)-3,3-Dimethyl-7-oxo-6-(2-phenylacetamido)-4-thia-1-azabicyclo[3.2.0]heptane-2-carboxylic acid compound with 2-(diethylamino) ethyl p-amino benzoate compound (1:1) monohydrate (Penicillin G procaine).

Bicillin C—R (Penicillin G benzathine and Penicillin G procaine suspension) is commercially available from Wyeth-Ayerst and is (2S,5R,6R)-3,3-Dimethyl-7-oxo-6-(2-phenylacetamido)-4-thia-1-azabicyclo[3.2.0]heptane-2-carboxylic acid compound with N,N′-dibenzylethylenediamine (2:1), tetrahydrate (Penicillin G benzathine) and (2S,5R,6R)-3,3-Dimethyl-7-oxo-6-(2-phenylacetamido)-4-thia-1-azabicyclo[3.2.0]heptane-2-carboxylic acid compound with 2-(diethylamino) ethyl p-amino benzoate compound (1:1) monohydrate (Penicillin G procaine).

Bicillin L-A (Penicillin G benzathine suspension) is commercially available from Wyeth-Ayerst and is (2S,5R,6R)-3,3-Dimethyl-7-oxo-6-(2-phenylacetamido)-4-thia-1-azabicyclo[3.2.0]heptane-2-carboxylic acid compound with N,N′-dibenzylethylenediamine (2:1), tetrahydrate.

Geocillin (carbencillin indanyl sodium) is commercially available from Pfizer and is 1-(5-Indanyl)-N-(2-carboxy-3-3-dimethyl-7-oxo-4-thia-1-azabicyclo [3.2.0]hept-6-yl)-2-phenylmalonamate monsodium salt.

Mezlin (sterile mezlocillin sodium) is commercially available from Bayer and is the monohydrate sodium salt of 6-{D-2[3-[(methyl-sulfonyl)-2-oxo-imidazolidine-1-carboxamido]-2-phenyl acetamido} penicillanic acid.

Omnipen (ampicillin) is commercially available from Wyeth-Ayerst and is (2S,5R,6R)-6-[(R)-2-Amino-2-phenylacetamido]-3,3-dimethyl-7-oxo-4-thia-1-aza-bicyclo[3.2.0]heptane-2-carboxylic acid.

Pen-Vee K (penicillin V potassium) is commercially available from Wyeth-Ayerst and is the potassium salt of the phenoxymethyl analog of penicillin G.

Pfizerpen (penicillin G potassium) is commercially available from Pfizer and is monopotassium 3,3-dimethyl-7-oxo-6-(2-phenylacetamido)-4-thia-1-azabicyclo (3.2.0) heptane-2-carboxylate.

Pipracil (piperacillin sodium) is commercially available from Lederle and is 4-thia-1-azabicyclo[3.2.0]heptane-2-carboxylic acid, 6-[[[[(4-ethyl-2-3-dioxo-1-piperazinyl) carbonyl]amino]phenylacetyl]amino]-3,3-dimethyl-7-oxo-, monosodium salt, [2S-[2α,5α,6β(S*)]].

Spectrobid (bacampicillin HCl) is commercially available from Pfizer and is 1′-ethoxycarbonyloxyethyl-6-(D-αaminophenylacetamide)-penicillate hydrochloride.

Ticar (ticarcillin disodium) is commercially available from SmithKline Beecham and is N-(2-carboxy-3,3-dimethyl-7-oxo-4-thia-1-azabicyclo[3.2.0]hept-6-yl)-3-thiophenemalonamic acid disodium salt.

Timentin (ticarcillin disodium and clavulanate potassium) is commercially available from SmithKline Beecham and is N-(2-carboxy-3,3-dimethyl-7-oxo-4-thia-1-azabicyclo[3.2.0]hept-6-yl)-3-thiophenemalonamic acid disodium salt (ticarcillin disodium) and potassium (Z)-(2R,5R)-3-(2-hydroxyethylidene)-7-oxo-4-oxa-1-azabicyclo[3.2.0]heptane-2-carboxylate (clavulanate potassium).

Unasyn (ampicillin sodium/sulbactam sodium) is commercially available from Pfizer and is monosodium (2S,5R,6R)-6-[(R)-2-Amino-2-phenyl acetamido]-3,3-dimethyl-7-oxo-4-thia-1-aza-bicyclo[3.2.0]heptane-2-carboxylate (amipicillin sodium), and sodium penicillate sulfone; sodium (2S,5R)-3,3-dimethyl-7-oxo-4-thia-1-azabicyclo[3.2.0]heptane-2-carboxylate 4,4-dioxide (sulbactam sodium).

Zosyn (piperacillin sodium and tazobactam sodium) is commercially available from Lederle and is sodium (2S,5R,6R)-6-[(R)-2-(4-ethyl-2,3-dioxo-1-piperazine-carboxamido)-2-phenylacetamido]-3,3-dimethyl-7-oxo-4-Thia-1-azabicylco-[3.2.0]heptane-2-carboxylate (piperacillin sodium), and sodium (2S,3S, 5R)-3-methyl-7-oxo-3-(1H-1,2,3-triazol-1-ylmethyl)-4-thia-1-azabicyclo[3.2.0]heptane-2-carboxylate-4,4-dioxide (tazobactam sodium).

Dicloxacillin Sodium is monosodium (2S,5R,6R)-6-(3-(2,6-dichlorophenyl)-5-methyl-4-isoxazolecarboxamido]-3,3-dimethyl-7-OXO-4-thia-1-azabicyclo[3.2.0]heptane-2-carboxylate monohydrate.

Achromycin V (tetracycline HCl) is commercially available from Lederle and is the monohydrochloride of [4S-(4α,4aα,5aα,6β,12aα)]-4-(Dimethylamino)-1,4,4a,5,5a,6,11,12a-octahydro-3,6,10,12,12a-pentahydroxy-6-methyl-1,11-dioxo-2-naphthacenecarboxamide.

Declomycin (demeclocycline HCl) is commercially available from Lederle Laboratories and is 7-chloro-4-dimethylamino)-1,4,4a,5,5a,6,11,12a-octahydro-3,6,10,12,12a-pentahydroxy-1,11-dioxo-2-naphthacenecarboxamide monohydrochloride.

Dynacin (minocylcine HCl) is commercially available from Medicis and is [4S-(4α,4aα,5aα,12aα)]-4,7-bis(dimethylamino)-1,4,4a,5,5a,6,11,12a-octahydro-3,10,12,12a-tetrahydroxy-1,11-dioxo-2-napthacene carboxamide monochloride.

Minocin (minocycline hydrochloride) is commercially available from Lederle Laboratories and is [4S-(4α,4aα,5aα,12aα)]-4,7-bis(dimethylamino)-1,4, 4a,5,5a,6,11,12a-octahydro-3,10,12,12a-tetrahydroxy-1,11-dioxo-2-napthacene carboxamide monochloride.

Monodox (Doxycycline monohydrate capsules) is commercially available from Oclassen and is α-6-deoxy-5-oxytetracycline.

Terramycin (oxytetracyline) is commercially available from Pfizer.

Vectrin (minocycline hydrochloride) is commercially available from Warner Chilcott Professional Products and is [4S-(4α,4aα,5aα,12ax)]-4,7-bis (dimethylamino)-1,4,4a,5,5a,6,11,12a-octahydro-3,10,12,12a-tetrahydroxy-1, 11-dioxo-2-napthacene carboxamide monochloride.

Vibramycin Calcium (doxycycline sodium) is commercially available from Pfizer and is 4-(Dimethylamino)-1,4,4a,5,5a,6,11,12a-octahydro-3,5,10,12, 12a-pentahydroxy-6-methyl-1,11-dioxo-2-napthacene-carboxamide monohydrate.

Vibramycin Hyclate (doxycycline hyclate) is commercially available from Pfizer and is α-6-deoxy-5-oxytetracycline.

Vibramycin Monohydrate (doxycycline monohydrate) is commercially available from Pfizer and is 4-(Dimethylamino)-1,4,4a,5,5a,6,11,12a-octahydro-3,5,10,12,12a-pentahydroxy-6-methyl-1,11-dioxo-2-napthacene-carboxamide monohydrate.

Vibra-Tabs (doxycycline hydrate) is commercially available from Pfizer and is α-6-deoxy-5-oxytetracycline.

Vibramycin (doxycycline) is commercially available from Pfizer and is 4-(Dimethylamino)-1,4,4a,5,5a,6,11,12a-octahydro-3,5,10,12,12a-pentahydroxy-6-methyl-1,11-dioxo-2-napthacene-carboxamide monohydrate.

Lincomycins is monosodium (2S,5R,6R)-6-(3-(2,6-dichlorophenyl)-5-methyl-4-isoxazolecarboxamido]-3,3-dimethyl-7-OXO-4-thia-1-azabicyclo[3.2.0]heptane-2-carboxylate monohydrate.

Cleocin HCl (clindamycin HCl) is commercially available from Pharmacia & Upjohn and is Methyl 7-chloro-6,7,8-trideoxy-6-(1-methyl-trans-4-propyl-L-2-pyrrolidine carboxamido)-1-thio-L-threo-α-D-galacto-octopyranoside monohydrochloride.

Abelcet (amphotericin B lipid complex) is commercially available from Libosome Company, Inc. and is [1R-(1R*, 3S*, 5R*, 6R*, 9R*, 11R*, 15S*, 16R*, 17R*, 18S*, 19E, 21E, 23E, 25E, 27E, 29E, 31E, 33R*, 35S*, 36R*, 37S*)]-33-[(3-amino-3,6-dideoxy-β-D-mannopyranosyl)-oxy]-1,3,5,6,9,11,17,37-octahydroxy-15,16,18-trimethyl-13-oxo-14,39-dioxabicyclo[33.3.1]nonatriaconta-19,21,23,25,27,29,31-heptaene-36-carboxylic acid.

AmBisome (amphotericin B) is commercially available from Fujisawa Healthcare and is [1R-(1R*, 3S*, 5R*, 6R*, 9R*, 11R*, 15S*, 16R*, 17R*, 18S*, 19E, 21E, 23E, 25E, 27E, 29E, 31E, 33R*, 35S*, 36R*, 37S*)]-33-[(3-amino-3,6-dideoxy-β-D-mannopyranosyl)-oxy]-1,3,5,6,9,11,17,37-octahydroxy-15,16, 18-trimethyl-13-oxo-14,39-dioxabicyclo[33.3.1]nonatriaconta-19,21,23,25,27, 29,31-heptaene-36-carboxylic acid.

Amphotec (amphotericin B cholesterol sulfate complex) is commercially available from Sequus Pharmaceuticals, Inc. and is [1R-(1R*, 3S*, 5R*, 6R*, 9R*, 11R*, 15S*, 16R*, 17R*, 18S*, 19E, 21E, 23E, 25E, 27E, 29E, 31E, 33R*, 35S*, 36R*, 37S*)]-33-[(3-amino-3,6-dideoxy-β-D-mannopyranosyl)-oxy]-1,3,5,6,9, 11,17,37-octahydroxy-15,16,18-trimethyl-13-oxo-14,39-dioxabicyclo[33.3.1]nonatriaconta-19,21,23,25,27,29,31-heptaene-36-carboxylic acid.

Ancobon (flucytosine) is commercially available from ICN Pharmaceuticals and is 5-fluorocytosine.

Diflucan (fluconazole) is commercially available from Pfizer Inc. and is 2, 4-difluoro-α-α′-bis(1H-1,2,4-triazol-1-ylmethyl)benzyl alcohol.

Fulvicin P/G (ultramicrosize griseofulvin) is commercially available from Schering.

Fulvicin P/G 165 and 330 (ultramicrosize griseofulvin) is commercially available from Schering.

Grifulvin V (griseofulvin) is commercially available from Ortho Dermatological.

Gris-PEG (griseofulvin ultramicrosize) is commercially available from Allergan.

Lamisil (terbinafine hydrochloride) is commercially available from Novartis and is (E)-N-(6,6-dimethyl-2-hepten-4-ynyl)-N-methyl-1-naphthalenemethanamine hydrochloride.

Nizoral (ketoconazole) is commercially available from Janssen and is cis 1-acetyl-4-[4-[[2-(2,4-di-chlorophenyl)-2-(1H-imidazol-1-ylmethyl)-1,3-dioxolan-4-yl]methoxy]phenyl]piperazine.

Amphotericin B is [1R-(1R*, 3S*, 5R*, 6R*, 9R*, 11R*, 15S*, 16R*, 17R*, 18S*, 19E, 21E, 23E, 25E, 27E, 29E, 31E, 33R*, 35S*, 36R*, 37S*)]-33-[(3-amino-3,6-dideoxy-β-D-mannopyranosyl)-oxy]-1,3,5,6,9,11,17,37-octahydroxy-15,16,18-trimethyl-13-oxo-14,39-dioxabicyclo[33.3.1]nonatriaconta-19,21,23,25,27,29,31-heptaene-36-carboxylic acid.

Lotrimin (clotrimazole) is commercially available from Schering and is 1-(O-Chloro-α,α-diphenyl benzyl)imidazole.

Dapsone tablets (dapsone) is commercially available from Jacobus and is 4, 4′-diaminodiphenyl-sulfone (DDS).

Diflucan (fluconazole) is commercially available from Pfizer and is 2,4-difluoro-α-α′-bis(1H-1,2,4-triazol-1-ylmethyl)benzyl alcohol.

Monistat-Derm cream (miconazole) is commercially available from Ortho Dermatological and is 1-[2,4-dichloro-β-{(2,4-dichlorobenzyl) oxy} phenethyl]imidazole mononitrate.

Mycostatin Cream (nystatin) is commercially available from Westwood-Squibb.

Sporanox (itraconazole) is commercially available from Janssen Pharmaceutical and is (±)-1-[(R*)-sec-butyl]-4-[p-[[2R*, 4S*)-2-(2,4-dichlorophenyl)-2-(1H-1,2,4-triazol-1-ylmethyl)-1,3-dioxolan-4-yl]methoxy]phenyl]-1-piperazinyl]phenyl]-Δ2-1,2,4, triazolin-5-one mixture with (±)-1-[(R*)-sec-butyl]-4-[p-[[2R*, 4S*)-2-(2,4-dichlorophenyl)-2-(1H-1,2,4-triazol-1-ylmethyl)-1,3-dioxolan-4-yl]methoxy]phenyl]-1-piperazinyl]phenyl]-Δ2-1,2,4, triazolin-5-one or (±)-1-[(RS)-sec-butyl]-4-[p-[4-[p-[[(2R,4S)-2-(2,4-dichlorophenyl)-2-(1H-1,2,3,4-triazol-1-ylmethy) 1,3-dioxolan-4-yl]methoxy]phenyl]-1-piperazinyl]phenyl]-Δ2-1,2,4, triazolin-5-one.

Aralen hydrochloride (chloroquine HCl) is commercially available from Sanofi Pharmaceuticals and is 7-(chloro-4-[[4-diethylamino)-1-methyl butyl]amino]-quinoline dihydrochloride.

Aralen phosphate (chloroquine phosphate) is commercially available from Sanofi Pharmaceuticals and is 7-(chloro-4-[[4-diethylamino)-1-methyl butyl]amino]-quinoline phosphate (1:2).

Daraprim (pyrimethamine) is commercially available from Glaxo Wellcome and is 5-(4-chlorophenyl)-6-ethyl-2,4-pyrimidinediamine.

Lariam (mefloquine HCl) is commercially available from Roche Laboratories and is (R*, S*)-(±)-α-2-piperidinyl-2,8-bis(trifluoromethyl)-4-quinoline methanol hydrochloride.

Plaquenil (hydroxychloroquine sulfate) is commercially available from Sanofi Pharmaceuticals and is 2-[[4-[7-chloro-4-quinolyl)amino]pentyl]ethylamino]ethanol sulfate (1:1).

Capastat sulfate (capreomycin sulfate) is commercially available from Dura Pharmaceuticals.

Myambutol (ethambutol hydrochloride) is commercially available from Lederle Laboratories.

Mycobutin (rifabutin capsules) is commercially available from Pharmacia & Upjohn and is 1′,4-didehydro-1-deoxy-1,4-dihydro-5′-(2-methylpropyl)-1-oxorifamycin XIV or (9S,12E,14S,15R,16S,17R,18R,19R,20S,21S,22E, 24Z)-6,16,18,20-tetrahydroxy-1-1′-isobutyl-[4-methoxy-7,9,15,17,19,21,25-heptamethyl-spiro[9,4-(epoxypentadeca[1,11,13]trienimino)-2H-furo[2′,3′:7,8]naphth[1,2-d]imidazole-2,4′-piperidine]-5,10,26-(3H, 9H)-trione-16-acetate.

Nydrazid (isoniazid injection) is commercially available from Apothecon.

Paser (aminosalicylic acid) is commercially available from Jacobus and is 4-amino-2-hydroxy benzoic acid.

Priftin (rifapentine) is commercially available from Hoechst Marion Roussel and is rifamycin 3-[[(4-cyclo-pentyl-1-piperazinyl) imino]methyl] or 3-[N-(4-cyclopentyl-1-piperazinyl)-formimyidoyl]-2,7-(epoxypentadeca[1,11,13]trienimino)naptho[2,1-b]furan-1,11 (2H)-dione 21-acetate.

Pyrazinamide tablets (pyrazinamide) is commercially available from Lederle Laboratories and is the pyrazine analogue of nicotinamide.

Rifadin (rifampin capsules) is commercially available from Hoechst Marion Roussel and is 3-[[(4-methyl-1-piperazinyl) imino]methyl]rifamycin or 5,6,9,17, 19,21-hexahydroxy-23-methoxy-2,4,12,16,20,22-heptamethyl-8-[N-methyl-1-piperazinyl) formimidoyl]-2,7-(epoxy pentadeca[1,11,13]trienimino)naptho[2,1-b]furan-1,11 (2H)-dione 21-acetate.

Rifadin IV (rifampin for injection) is commercially available from Hoechst Marion Roussel and is 3-[[3-(4-methyl-1-piperazinyl) formimidoyl]-2,7-(epoxy pentadeca[1,11,13]trienimino) naphtho[2,1-b]furan-1,11 (2H)-dione 21-acetate.

Rifamate (rifampin and isoniazid) is commercially available from Hoechst Marion Roussel and is 3-(4-methyl-1-piperazinyliminomethyl) rifamycin SV (rifampin) and hydrazide of isonicotinic acid (isoniazid).

Rifater (rifampin, isoniazid and pyrazinamide) is commercially available from Hoechst Marion Roussel and is 3-(4-methyl-1-piperazinyliminomethyl) rifamycin SV (rifampin), hydrazide of isonicotinic acid (isoniazid), and pyrazine analogue of nicotinamide (pyrazinamide).

Seromycin (cycloserine capsules) is commercially available from Dura Pharmaceuticals and is 3-isoxazolidinone, 4-amino-, (R)—.

Streptomycin Sulfate is commercially available from Pfizer and is O-2-deoxy-2-(methylamino)-α-L-glyucopyransoyl-(1□2)-O-5-deoxy-3-C-formyl-α-L-lyxofuranosyl-(1□4)—N—N′-bis(aminoiminomethyl)-, sulfate (2:3) salt.

Tice BCG (BCG vaccine) is commercially available from Organon and is attenuated live Mycobacterium bovis strains Bacillus of Calmette and Guerin.

Cycloserine (seromycin capsules) is commercially available from Dura Pharmaceuticals and is 3-isoxazolidinone, 4-amino-, (R)—.

Nydrazid (Isoniazid) is commercially available from Apothecon and is the hydrazide of isonicotinic acid.

Urised (Methenamine) is commercially available from Poly Medica.

Trecator-SC (ethionamide tablets) is commercially available from Wyeth-Ayerst and is 2-ethylthioisonicotinamide.

Alferon N (interferon alfa-n3) is commercially available from Interferon Sciences and is interferon alfa-n3 (human leukocyte derived).

Crixivan (indinavir sulfate) is commercially available from Merck & Co., Inc. and is [1(1S,2R), 5(S)]-2,3,5-trideoxy-N-(2,3-dihydro-2-hydroxy-1H-inden-1-yl)-5-[2-[[1,1-dimethylethyl)amino]carbonyl]-4-(3-pyridinyl-methyl)-1-piperazinyl]-2-(phenylmethyl)-D-erythropentonamide sulfate (1:1).

Cytovene (ganciclovir) is commercially available from Roche and is 9-[[2-hydroxy-1 (hydroxymethyl)ethoxy]methyl]guanine.

Cytovene-IV (ganciclovir sodium) is commercially available from Roche and is 9-[[2-hydroxy-1 (hydroxymethyl)ethoxy]methyl]guanine.

Epivir (lamivudine) is commercially available from Glaxo Wellcome and is (2R, cis)-4-amino-1-(2-hydroxymethyl-1,3-oxathiolan-5-yl)-1H)-pyrimidin-2-one.

Famvir (famciclovir) is commercially available from SmithKline Beecham and is 2-[2-(2-amino-9H-purin-9-yl)ethyl]-1,3-propanediol diacetate.

Flumadine (rimantadine HCl) is commercially available from Forest and is alpha-methyltricyclo-[3.3.1.1/3.7]decane-1-methanamine hydrochloride.

Foscavir (foscarnet sodium) is commercially available from Astra and is phosphonoformic acid, trisodium salt.

Hivid (zalcitabine) is commercially available from Roche and is 4-amino-1-beta-D-2′,3′, dideoxyribofuranosyl-2-(1H)-pyrimidone or 2′,3′-dideoxycytidine.

Intron A (interferon alfa-2b) is commercially available from Schering.

Invirase (saquinavir mesylate) is commercially available from Roche Labs and is N-tert-butyl-decahydro-2-[2(R)-hydroxy-4-phenyl-3(S)-[[N-(2-quinolylcarbonyl)-L-asparaginyl]amino]butyl-(4aS,8aS)-isoquinoline-3(S)-carboxamide methanesulfonate.

Norvir (ritonavir) is commercially available from Abbott and is 10-Hydroxy-2-methyl-5-(1-methylethyl)-1-[2-(1-methylethyl)-4-thiazolyl]-3,6-dioxo-8,11-bis(phenylmethyl)-2,4,7,12-tetraazamidencan-B-oic acid, 5-thiazolyl methyl ester [5S-(5R*, 8R*, 10R*, 11R*)].

Rebetron combination therapy, which contains Rebetrol (ribavirin which is 1-β-D-ribofuranosyl-1H-1,2,4-triazole-3-carboxamide) and Intron A (inteferon alfa-2b), is commercially available from Schering.

Rescriptor (delavirdine mesylate) is commercially available from Pharmacia & Upjohn and is pieperazine, 1-[3-[(1-methylethyl)amino]-2-pyridinyl]-4-[[5(methylsulfonyl)-amino]-1H-indol-2-yl]carbonyl], monomethanesulfonate.

Retrovir (ziduvudine) is commercially available from Glaxo Wellcome and is 3′-azido-3′-deoxythymidine.

Retrovir IV (zidovudine) is commercially available from Glaxo-Wellcome and is 3′-azido-3′-deoxythymidine.

Symmetrel (amantadine hydrochloride) is commercially available from Endo Pharmaceuticals and is 1-adamantanamine hydrochloride.

Synagis (palivizumab) is commercially available from MedImmune Inc. and is humanized monoclonal antibody (IgG1κ).

Valtrex (valacyclovir HCl) is commercially available from Glaxo Wellcome and is L-valine, 2-[(2-amino-1,6-dihydro-6-oxo-9H-purin-9-yl)methoxy]ethyl ester, monohydrochloride.

Videx (didanosine) is commercially available from Bristol-Myers Squibb Oncology/Immunology and is 2′,3′-di-deoxyinosine.

Viracept (nelfinavir mesylate) is commercially available from Agouron and is [3S-[2(2S*, 3S*), 3α,4aβ,8aβ]]-N-(1,1-dimethylethyl)decahydro-2-[2-hydroxy-3-[(3-hydroxy-2-methylbenzoyl)amino]-4-(phenylthio)butyl]-3-isoquinolinecarboxcamide mono-methanesulfonate (salt).

Viramune (nevirapine) is commercially available from Roxane and is 11-cyclopropyl-5,11-dihydro-4-methyl-6H-dipyrido[3,2-b:2′,3′-][1,4]diazepin-6-one.

Virazole (ribavirin) is commercially available from ICN and is 1-beta-D-ribofuranosyl-1H-1,2,4-triazole-3-carboxamide.

Vistide (cidofovir) is commercially available from Gilead Sciences and is 1-[(S)-3-hydroxy-2-(phosphonomethoxy)propyl]cytosine dihydrate (HPMPC).

Zerit (stavudine (d4T)) is commercially available from Bristol-Myers Squibb Oncology/Immunology and is 2′,3′-didehydro-3′deoxythymidine.

Symmetrel Syrup (amantadine HCl) is commercially available from Endo Labs and is 1-adamantanamine hydrochloride.

Combivir Tablets (lamiduvine) is commercially available from Glaxo Wellcome and is 2′,3′-didehydro-3′-deoxythymidine.

Zovirax (acyclovir) is commercially available from Glaxo Wellcome and is 2-amino-1,9-dehydro-9-[(2-hydroxyethyoxy)methyl]-6H-purin-6-one.

Dapsone Tablets (dapsone) is commercially available from Jacobus and is 4,4′-diaminodiphenylsulfone (DDS).

Daraprim (pyrimethamine) is commercially available from Glaxo Wellcome and is 5-(4-chlorophenyl)-6-ethyl-2,4-pyrimidinediamine.

Flagyl 375 (metronidazole) is commercially available from Searle and is 2-Methyl-5-nitro-imidazole-1-ethanol.

Flagyl ER Tablets (metronidazole) is commercially available from Searle and is 2-Methyl-5-nitro-imidazole-1-ethanol.

Flagyl I.V. (metronidazole) is commercially available from SCS and is 2-Methyl-5-nitro-imidazole-1-ethanol.

Furoxone (furazolidone) is commercially available from Roberts and is 3-(5-nitrofurfuryliden-amino)-2-oxazolidinone.

Mepron (atovaquone) is commercially available from Glaxo Wellcome and is trans-2-[4-(4-chlorophenyl)cyclohexyl]-3-hydroxy-1,4-naphthalenedione.

Neutrexin (trimetrexate glucuronate) is commercially available from U.S. Bioscience and is 2,4-diamino-5-methyl-6-[(3,4,5-trimethoxyanilino)methyl]quinazoline mono-D-glucuronate.

Cipro (ciprofloxacin HCl) is commercially available from Bayer and is the monohydrochloride monohydrate salt of 1-cyclopropyl-6-fluoro-1,4-dihydro-4-oxo-7-(1-piperazinyl)-3-quinolinecarboxylic acid.

Floxin (ofloxacin) is commercially available from Ortho-McNeil Pharmaceutical and is (±)-9-fluoro-2,3-dihydro-3-methyl-10-(4-methyl-1-piperazinyl)-7-oxo-7H-pyrido[1,3,3-de]-1,4-benzoxazine-6-carboxylic acid.

Levaquin (levofloxacin) is commercially available from Ortho-McNeil Pharmaceutical) and is (−)-(S)-9-fluoro-2,3-dihydro-3-methyl-10-(4-methyl-1-piperazinyl)-7-oxo-7H-pyrido[1,2,3-de]-1,4-benzoxazine-6-carboxylic acid hemihydrate.

Mazaquin (lomefloxacin HCl) is commercially available from Unimed and is monohydrochloride salt of (±)-1-ethyl-6,8-difluoro-1,4-dihydro-7-(3-methyl-1-piperazinyl)-4-oxo-3-quinolinecarboxylic acid.

Noroxin (norfloxacin) is commercially available from Merck and is 1-ethyl-6-fluoro-1,4-dihydro-4-oxo-7-(1-piperazinyl)-3-quinolinecarboxylic acid.

Penetrex (enoxacin) is commercially available from Rhône-Poulenc Rorer and is 1-ethyl-6-fluoro-1,4-dihydro-4-oxo-7-(1-piperazinyl)-1,8-naphthyridine-3-carboxylic acid sesquihydrate.

Raxar (grepafloxacin HCl) is commercially available from Glaxo Wellcome and is (±)-1-cyclopropyl-6-fluoro-1,4-dihydro-5-methyl-7-(3-methyl-1-piperazinyl)-4-oxo-3-quinolinecarboxylic acid monochloride sesquihydrate.

Trovan (trovafloxacin mesylate) is commercially available from Pfizer and is (1α,5α,6a)-7-(6-amino-3-azabicyclo[3.1.0]hex-3-yl)-1-(2,4-difluorophenyl)-6-fluoro-1,4-dihydro-4-oxo-1,8-naphthyridine-3-carboxylic acid, monomethanesulfonate.

Zagam (sparfloxacin) is commercially available from Rhône-Poulenc Rorer and is 5-Amino-1-cyclopropyl-7-cis-3,5-dimethyl-1-piperazinyl)-6,8-difluoro-1,4, dihydro-4-oxo-3-quinolinecarboxylic acid.

Bactrim (trimethoprim and sulfamethoxazole) is commercially available from Roche Labs and is 2,4-diamino-5-(3,4,5-trimethoxybenzyl)pyrimidine (trimethoprim) and N1,-(5-methyl-3-isoxazolyl)sulfanilamide (sulfamethoxazole).

Bactrim DS (trimethoprim and sulfamethoxazole double strength) is commercially available from Roche Labs and is 2,4-diamino-5-(3,4,5-trimethoxybenzyl)pyrimidine (trimethoprim) and N1,-(5-methyl-3-isoxazolyl)sulfanilamide (sulfamethoxazole).

Pediazole (erythromicin ethylsuccinate and sulfisaxazole acetyl) is commercially available from Ross and is erythromicin 2′-(ethyl succinate) and N′ acetyl sulfisoxazole (sulfisoxizole is N-(3,4-Dimethyl-5-isoxazolyl)-N-sulfanilyl acetamide.

Septra (trimethoprim and sulfamethoxazole) is commercially available from Monarch and is 5-[(3,4,5-trimethoxyphenyl)methyl]-2,4-pyrimidinediamine (trimethoprim) and 4-amino-N-(5-methyl-3-isoxazolyl)benzenesulfonamide (sulfamethoxazole).

Septra DS (trimethoprim and sulfamethoxazole) is commercially available from Monarch and is 5-[(3,4,5-trimethoxyphenyl)methyl]-2,4-pyrimidinediamine (trimethoprim) and 4-amino-N-(5-methyl-3-isoxazolyl)benzenesulfonamide (sulfamethoxazole).

Co-trimoxazole is a combined chemotherapeutic agent consisting of trimethoprim (T) and the sulphonamide sulphamethoxazole (S); their ratio is 1:5. It is bactericidal by virtue of a sequential blockade of the folic acid synthesis in micoorganisms. The antimicrobial spectrum of co-trimoxazole includes many Gram-positive and Gram-negative aerobes, Chlamydias, nocardias, protozoas (pneumocystis carinii), etc. In addition to its use for pneumocystis, co-trimoxazole mainly has practical importance against Gram-positive aerobes (urinary tract infections), pneumococci, and haemophilus influenzae (respiratory tract infections and otitis). http://www.infomed.org/100drugs/ctrifram.html.

Bactrim I.V. Infusion (sulfamethoxazole) is commercially available from Roche Labs.

Pediazole (erythromicin ethylsuccinate and sulfisoxazole acetyl) is commercially available from Ross and is erythromicin 2′-(ethyl succinate) and N′ acetyl sulfisoxazole (sulfisoxizole is N-(3,4-Dimethyl-5-isoxazolyl)-N-sulfanilyl acetamide.

Furadantin (nitrofurantoin) is commercially available from Dura and is 1-[[(5-nitro-2-furanyl)methylene]amino]-2,4-imidazolidinedione.

Macrobid (nitrofurantoin monohydrate macrocrystals) is commercially available from Procter & Gamble Pharmaceuticals and is 1-[[[5-nitro-2-furanyl]methylene]amino]-2-4-imidazolidinedione monohydrate.

Macrodantin (nitrofurantoin macrocrystals) is commercially available from Procter & Gamble Pharmaceuticals and is 1-[[[5-nitro-2-furanyl]methylene]amino]-2-4-imidazolidinedione.

Monurol Sachet (fosfomycin tromethamine) is commercially available from Forest and is (1R,2S)-(1,2-epoxypropyl) phosphonic acid, compound with 2-amino-2-(hydroxymethyl)-1,3-propanediol (1:1).

NegGram Caplets (nalidixic acid) is commercially available from Sanofi and is 1-ethyl-1,4-dihydro-7-methyl-4-oxo-1,8-naphthyridine-3-carboxylic acid.

Septra (trimethoprim and sulfamethoxazole) is commercially available from Monarch and is 5-[(3,4,5-trimethoxyphenyl)methyl]-2,4-pyrimidinediamine (trimethoprim) and 4-amino-N-(5-methyl-3-isoxazolyl)benzenesulfonamide (sulfamethoxazole).

Septra DS (trimethoprim and sulfamethoxazole) is commercially available from Monarch and is 5-[(3,4,5-trimethoxyphenyl)methyl]-2,4-pyrimidinediamine (trimethoprim) and 4-amino-N-(5-methyl-3-isoxazolyl)benzenesulfonamide (sulfamethoxazole).

Urised (a combination of the antiseptics methenamine, methylene blue, phenyl salicylate, benzoic acid and parasympatholytics (atropine sulfate) hyoscyamine) is commercially available from Poly Medica.

Urobiotic-250 Capsules (oxytetracycline HCl, sulfamethizole and phenazopyridine HCl) is commercially available from Pfizer.

Uroqid Acid No. 2 Tablets (methenamine mandelate) is commercially available from Beach.

Bactroban (mupirocin) is commercially available from SmithKline Beecham and is (αE, 2S, 3R,4R,5S)-5-[(2S,3S,4S,5S)-2,3-Epoxy-5-hydroxy-4-methylhexyl]tetrahydro-3,4-dihydroxy-β-methyl-2H-pyran-2-crotonic acid, ester with 9-hydroxynonanoic acid, calcium salt (2:1), dihydrate.

Chloromycetin ophthalmic (chloramphenical) is commercially available from Monarch and is (1) Acetamide, 2,2-dichloro-N-[2-hydroxy-1-(hydroxymethyl)-2-(4-nitrophenyl)ethyl]-, and (2) D-threo-(−)-2,2-Dichloro-N-[□-hydroxy-□-(hydroxymethyl)-p-nitrophenethyl]acetamide.

Cortisporin (neomycin and polymyxin β sulfates and hydrocortisone acetate cream) is commercially available from Monarch and is 21-(acetyloxy)-11β,17-dihydroxypregn-4-ene-3,20-dione.

Ilotycin (erythromycin ophthalmic ointment) is commercially available from Dista and is (3R*, 4S*, 5S*, 6R*, 7R*, 9R*, 11R*, 12R*, 13S*, 14R*)-4-[(2,6-dideoxy-3-C-methyl-3-O-methyl-α-L-ribo-hexopyranosyl) oxy]-14-ethyl-7,12,13-trihydroxy-3,5,7,9,11,13, hexamethyl-6-[[3,4,6-trideoxy-3-(dimethylamino)-β-D-xylo-hex-opyranosyl]oxy]oxacyclotetradecane-2,10-dione.

NeoDecadron (neomycin sulfate-dexamethasone sodium phosphate) is commercially available from Merck and is 9-fluoro-11β,17-dihydroxy-16a-methyl-21-(pbosphonooxy)pregna-1,4-diene-3,20-dione disodium salt.

Polytrim (trimethoprim and polythyxin β sulfate ophthalmic solution) is commercially available from Allergan and is 2,4-diamino-5-(3,4,5-trimethoxylbenzl)pyrimidine (trimethoprim) and the sulfate salt of polymyxin B1 and B2 (polythyxin β sulfate).

Terra-Cortril (oxytetracycline HCl and hydrocortisone acetate) is commercially available from Pfizer.

TobraDex (tobramycin and dexamethasone ophthalmic suspension and ointment) is commercially available from Alcon and is O-3-Amino-3-deoxy-a-D-glucopyranosyl-(1□4)—O—[2,6-diamino-2,3,6-trideoxy-a-D-ribo-hexopyranosyl-1(1□6)]-2-deoxy-L-streptamine. Dexamethasone: Chemical Name: 9-Fluoro-11b, 17,21-trihydroxy-16a-methylpregna-1,4-diene-3,20-dione.

Vira-A ophthalmic ointment, 3% (vidarabine) is commercially available from Monarch and is 9H-Purin-6-amine, 9-β-D-arabinofuranosyl-, monohydrate.

Chibroxin (norfloxacin ophthalmic solution) is commercially available from Merck and is 1-ethyl-6-fluoro-1,4-dihydro-4-oxo-7-(1-piperazinyl)-3-quinolinecarboxylic acid.

Ciloxan ophthalmic solution, (Ciprofloxacin HCl) is commercially available from Alcon and is the monohydro chloride monohydrate salt of 1-cyclopropyl-6-fluoro-1,4-dihydro-4-oxo-7-(1-piperazinyl)-3-quinoline-carboxylic acid.

Ciloxan ophthalmic ointment, (Ciprofloxacin HCl) is commercially available from Alcon and is the monohydro chloride monohydrate salt of 1-cyclopropyl-6-fluoro-1,4-dihydro-4-oxo-7-(1-piperazinyl)-3-quinoline-carboxylic acid.

Ocuflox ophthalmic solution (ofloxacin) is commercially available from Allergan and is (±)-9-Fluoro-2,3-dihydro-3-methyl-10-(4-methyl-1-piperazinyl)-7-oxo-7H-pyrido[1,2,3-de]-1,4 benzoxazine-6-carboxylic acid.

Blephamide ophthalmic ointment (sulfacetamide sodium and prednisolone acetate) is commercially available from Allergan and is N-sulfanilyl-acetamide monosodium salt monohydrate (sulfacetamide sodium) and 11β,17,21-trihydroxypreyna-1,4-diene-3,20-dione 21-acetate (prednisolone acetate).

Blephamide ophthalmic suspension (sulfacetamide sodium and prednisolone acetate) is commercially available from Allergan and is N-sulfanilyl-acetamide monosodium salt monohydrate (sulfacetamide sodium) and 11β,17,21-trihydroxypreyna-1,4-diene-3,20-dione 21-acetate (prednisolone acetate).

A/T/S (erythromycin) is commercially available from Hoescht Marion Roussel and is (3R*, 4S*, 5S*, 6R*, 7R*, 9R*, 11R*, 12R*, 13S*, 14R*)-4-[(2,6-dideoxy-3-C-methyl-3-O-methyl-α-ribo-hexopyranosyl) oxy]-14-ethyl-7,12,13-trihydroxy-3,5,7,9,11,13, hexamethyl-6-[[3,4,6-trideoxy-3-(dimethylamino)-β-D-xylo-hex-opyranosyl]oxy]oxacyclotetradecane-2,10-dione.

Bactroban (mupirocin) is commercially available from SKB and is (αE, 2S, 3R,4R,5S)-5-[(2S,3S,4S,5S)-2,3-Epoxy-5-hydroxy-4-methylhexyl]tetrahydro-3,4-dihydroxy-β-methyl-2H-pyran-2-crotonic acid, ester with 9-hydroxynonanoic acid, calcium salt (2:1), dihydrate.

Benzamycin (erythromycin-benzoyl peroxide topical gel) is commercially available from Dermik and is (3R*, 4S*, 5S*, 6R*, 7R*, 9R*, 11R*, 12R*, 13S*, 14R*)-4-[(2,6-dideoxy-3-C-methyl-3-O-methyl-α-L-ribo-hexopyranosyl) oxy]-14-ethyl-7,12,13-trihydroxy-3,5,7,9,11,13, hexamethyl-6-[[3,4,6-trideoxy-3-(dimethylamino)-β-D-xylo-hex-opyranosyl]oxy]oxacyclotetradecane-2,10-dione (erythromycin).

Betadine (povidone-iodine) is commercially available from Purdue Frederick.

Cleocin T (clindamycin phosphate topical solution) is commercially available from Pharmacia & Upjohn and is L-threo-I-D-galacto-Octopyranoside, methyl 7-chloro-6,7,8-trideoxy-6-[[(1-methyl-4-propyl-2-pyrrolidinyl)-carbonyl]amino]-1-thio-, 2-(dihydrogen phosphate), (2S-trans)-.

Clindets (clindamycin phosphate pledgets) is commercially available from Stiefel and is methyl 7-chloro-6,7,8-trideoxy-6-(1-methyl-trans-4-propyl-L-2-pyrrolidinecarboxamido)-1-thio-L-threo-□-D-galacto-octopyranoside 2-(dihydrogen phospate).

Emgel (erythromycin) is commercially available from Glaxo Wellcome and is (3R*, 4S*, 5S*, 6R*, 7R*, 9R*, 11R*, 12R*, 13S*, 14R*)-4-[(2,6-Dideoxy-3-C-methyl-3-O-methyl-α-L-ribo-hexopyranosyl)oxy]-14-ethyl-7,12,13-trihydroxy-3,5,7,9,11,13-hexamethyl-6-[[3,4,6-trideoxy-3-(dimethyl-amino)-β-D-xylo-hexopyranosyl]oxy]oxacyclotetradecane-2,10-dione.

Erycette (erythromycin topical solution) is commercially available from Ortho Dermatological and is (3R*, 4S*, 5S*, 6R*, 7R*, 9R*, 11R*, 12R*, 13S*, 14R*)-4-[(2,6-Dideoxy-3-C-methyl-3-O-methyl-α-L-ribo-hexopyranosyl)oxy]-14-ethyl-7,12,13-trihydroxy-3,5,7,9,11,13-hexamethyl-6-[[3,4,6-trideoxy-3-(dimethyl-amino)-β-D-xylo-hexopyranosyl]oxy]oxacyclotetradecane-2,10-dione.

Klaron (sodium sulfacetamide lotion) is commercially available from Dermik.

Mycostatin (nystatin cream) is commercially available from Westwood-Squibb.

Theramycin Z (erythromycin topical solution) is commercially available from Medicis and is (3R*, 4S*, 5S*, 6R*, 7R*, 9R*, 11R*, 12R*, 13S*, 14R*)-4-[(2,6-dideoxy-3-C-methyl-3-O-methyl-α-L-ribo-hexopyranosyl) oxy]-14-ethyl-7, 12,13-trihydroxy-3,5,7,9,11,13, hexamethyl-6-[[3,4,6-trideoxy-3-(dimethylamino)-β-D-xylo-hex-opyranosyl]oxy]oxacyclotetradecane-2,10-dione.

T-Stat (erythromycin) is commercially available from Westwood-Squibb and is (3R*, 4S*, 5S*, 6R*, 7R*, 9R*, 11R*, 12R*, 13S*, 14R*)-4-[(2,6-dideoxy-3-C-methyl-3-O-methyl-α-L-ribo-hexopyranosyl) oxy]-14-ethyl-7,12,13-trihydroxy-3,5,7,9,11,13, hexamethyl-6-[[3,4,6-trideoxy-3-(dimethylamino)-β-D-xylo-hex-opyranosyl]oxy]oxacyclotetradecane-2,10-dione.

Exelderm (sulconazole nitrate) is commercially available from Westwood-Squibb and is (±)-1-[2,4-dichloro-β-[(p-chlorobenzyl)-thio]-plenethyl]imidazole mononitrate;

Fungizone (amphotericin B oral suspension) is commercially available from Bristol-Myers Squibb and is [1R-(1R*,3S*,5R*,6R*,9R*,11R*, 15S*,16R*,17R*,18S*,19E,21E,23E,25E,27E,29E,31E,33R*,35S*,36R*,37S*)]-33-[(3-Amino-3,6-dideoxy-β-D-mannopyranosyl)-oxy]-1,3,5,6,9,11,17,37-octahydroxy-15,16,18-trimethyl-13-oxo-14,39-dioxabicyclo[33.3.1]nonatriaconta-19,21,23,25,27,29,31-heptaene-36-carboxylic acid.

Lamisil (terbinafine hydrochloride cream) is commercially available from Novartis and is (E)-N-(6,6-dimethyl-2-hepten-4-ynyl)-N-methyl-1-naphthalenemethanamine hydrochloride.

Loprox (ciclopiroxolamine) is commercially available from Hoescht Marion Roussel and is 6-cyclohexyl-1-hydroxy-4-methyl-2(1H)-pyridone, 2-amino-ethanol salt.

Lotrimin (clotrimazole) is commercially available from Schering and is 1-(O-Chloro-α,α-diphenyl benzyl)imidazole.

Lotrisone (clotrimazole and betamethasone diproprionate) is commercially available from Schering and is 1-(O-Chloro-α,α-diphenyl benzyl)imidazole (clotrimazole) and 9-Fluoro-11β,17,21-trihroxy-16β-methylpregna-1,4-diene-3,20-dione 17,21-diproprionate (betamethasone diproprionate).

Mentax (butenafine HCl) is commercially available from Penederm and is N-4-tert-butylbenzyl-N-methyl-1-naphthalenemethylamine hydrochloride.

Monistat-Derm (miconazole nitrate) is commercially available from Ortho Dermatological and is 1-[2,4-dichloro-β-{(2,4-dichlorobenzyl)oxy)}phenethyl]imidazole mononitrate.

Mycelex (clotrimazole) is commercially available from Alza and is [1-(o-chloro-α, α-diphenylbenzyl) imidazole.

Mycostatin (nystatin) is commercially available from Westwood-Squibb.

Naftin (naftifine HCl) is commercially available from Allergan and is (E)-N-Cinnamyl-N-methyl-1-naphthalene-methylamine hydrochloride.

Nizoral (ketoconazole) is commercially available from Janssen and is cis-1-acetyl-4[4-[[2-(2,4-dichorophenyl)-2-(1H-imidazol-1-ylmethyl)-1,3-dioxolan-4-yl]methoxy]phenyl]piperazine.

Nystop (nystatin) is commercially available from Paddock.

Oxistat (oxiconazole nitrate) is commercially available from Glaxo Wellcome and is 2′,4′-dichloro-2-imidazole-1-ylacetophenone (Z)-[O-(2,4-dichlorobenzyl)oxime], mononitrate.

Selsun Rx (2.5% selenium sulfide lotion) is commercially available from Ross.

Spectazole (econazole nitrate) is commercially available from Ortho Dermatological and is 1-[2-{(4-chorophenyl)methoxy}-2-(2,4-dichlorophenyl)-ethyl]-1H-imidazole mononitrate.

Denavir (penciclovir cream) is commercially available from SmithKline Beecham and is 9-[4-hydroxy-3-(hydroxymethyl)butyl]guanine.

Zovirax (acyclovir) is commercially available from Glaxo-Wellcome and is 2-amino-1,9-dihydro-9-(2-hydroxyethoxy)methyl-6H-purin-6-one.

Benzashave (benzoyl peroxide) is commercially available from Medicis.

Betadine (povidone-iodine) is commercially available from Purdue Frederick.

Betasept (chlorhexidine gluconate) is commercially available from Purdue Frederick.

Cetaphil (soap substitute) is commercially available from Galaderma.

Clorpactin WCS-90 (sodium oxychlorosene) is commercially available from Guardiam Laboratories.

Dapsone Tablets (dapsone) is commercially available from Jacobus and is 4,4′-diaminodiphenyl sulfone (DDS).

Desquam-E (benzoyl peroxide) is commercially available from Westwood-Squibb.

Desquam-X (benzoyl peroxide) is commercially available from Westwood-Squibb.

Hibiclens (chlorhexidine gluconate) is commercially available from Zeneca.

Hibistat (chlorhexidine gluconate) is commercially available from Zeneca.

Impregon (tetrachlorosalicylanilide 2%) is commercially available from Fleming.

MetroCream (metronidazole) is commercially available from Galaderma and is 2-methyl-5-nitro-1H-imidazole-1-ethanol.

MetroGel (metronidazole) is commercially available from Galaderna and is 2-methyl-5-nitro-1H-imidazole-1-ethanol.

Noritate (metronidazole) is commercially available from Dermik and is 2-methyl-5-nitro-1H-imidazole-1-ethanol.

pHisoHex (hexachlorophene detergent cleanser) is commercially available from Sanofi and is Phenol,2,2′-methylene-bis[3,4,6-trichloro-].

Sulfacet-R (sodium sulfacetamide 10% and sulfur 5%) is commercially available from Dermik.

Sulfamylon (matenide acetate) is commercially available from Bertek and is α-amino-ρ-toluenesulfonamide monoacetate.

Triaz (benzoyl peroxide) is commercially available from Medicis.

Vanoxide-HC (benzoyl peroxide hydrocortisone) is commercially available from Dermik and is 11β,17,21-trihydroxypregn-4-ene-3,20-dione (hydrocortisone).

Acticin (perrnethrin) is commercially available from Penederm and is (±)-3-phenoxybenzyl 3-(2,2-dichlorovinyl)-2,2-dimethylcyclopropanecarboxylate.

Elimite (permethrin) is commercially available from Allergan and is (±)-3-phenoxybenzyl 3-(2,2-dichlorovinyl)-2,2-dimethylcyclopropanecarboxylate.

Eurax (crotamiton) is commercially available from Westwood-Squibb and is N-ethyl-N-(o-methylphenyl)-2-butenamide.

Lindane Lotion USP 1% (lindane) is commercially available from Alpharma.

Efudex (fluorouracil) is commercially available from ICN and is 5-fluoro-2,4 (1H, 3H)-pyrimidinedione.

Fluoroplex (fluorouracil) is commercially available from Allergan and is 5-fluoro-2,4 (1H, 3H)-pyrimidinedione.

Furadantin Oral Suspension (nitrofurantoin) is commercially available from Dura and is 1-[[5-nitro-2-furanyl)methylene]amino]-2,4-imidazolidine dione.

Zyvox (linezolid) is commercially available from Pharmacia & Upjohn.

It is appreciated that those skilled in the art understand that the antibiotic useful in the present invention is the biologically active compound present in any of the antibiotic drugs disclosed above. For example, Azactam (aztreonam) is typically available as an injectable solution. The antibiotic, however, is (z)-2-[[[(2-amino-4-thiazolyl) [[(2S,-3S)-2-methyl-4-oxo-1-sulfo-3-azetidinyl]carbamoyl]methylene]amino]oxy]-2-methyl propionic acid. Physician's Desk Reference (PDR), Medical Economics Company (Montvale, N.J.), (53rd Ed.), pp. 820-823, 1999.

### Anti-Inflammatory Steroid

Steroidal anti-inflammatory agents, including but not limited to, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortisone butylesters, fluocortolone, fluprednidene (fluprednidene) acetate, flurandrenolone, halcinonide, hydrocortisone acetate, hydrocortisone butyrate, methylprednisolone, triamcinolone acetonide, cortisone, cortodoxone, fluocinonide, fludrocortisone, diflorasone diacetate, flurandrenolone, fludrocortisone, diflorasone diacetate, flurandrenolone acetonide, medrysone, amcinafel, amcinonide, betamethasone and the balance of its esters, chlorotrianisene, chlorotrianisene acetate, clocortolone, clescinolone, dichlorisone, difluprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used. In some embodiments, the steroidal anti-inflammatory used is hydrocortisone.

The steroidal anti-inflammatory agent can be present in any suitable, safe and effective amount. For example, the steroidal anti-inflammatory agent can be present in up to about 5.0 wt. % of the composition of the present invention, up to about 2.0 wt. % of the composition of the present invention, or up to about 1.0 wt. % of the composition of the present invention.

### Non-Steroidal Anti-Inflammatory Drug (NSAID)

A second class of anti-inflammatory agents that is useful in the compositions includes the nonsteroidal anti-inflammatory agents. A variety of compounds are encompassed by this group. For detailed disclosure of the chemical structure, synthesis, side effects, etc. of non-steroidal anti-inflammatory agents, one may refer to standard texts, including Anti-inflammatory and Anti-Rheumatic Drugs, K. D. Rainsford, Vol. I-III, CRC Press, Boca Raton, (1985), and Anti-inflammatory Agents, Chemistry and Pharmacology, 1, R. A. Scherrer, et al., Academic Press, New York (1974).

Specific non-steroidal anti-inflammatory agents useful in the composition invention include, but are not limited to:

(1) the oxicams, such as piroxicam, isoxicam, tenoxicam, sudoxicam, and CP-14,304;

(2) benorylate, trilisate, safapryn, solprin, diflunisal, and fendosal;

(3) the acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac;

(4) the fenamates, such as mefenamic, meclofenamic, flufenamic, niflumic, and tolfenamic acids;

(5) the propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and

(6) the pyrazoles, such as phenylbutazone, oxyphenbutazone, feprazone, azapropazone, and trimethazone.

Mixtures of these non-steroidal anti-inflammatory agents may also be employed, as well as the dermatologically acceptable salts and esters of these agents. For example, etofenamate, a flufenamic acid derivative, is useful for topical application. Of the nonsteroidal anti-inflammatory agents, ibuprofen, naproxen, flufenamic acid, etofenamate, aspirin, mefenamic acid, meclofenamic acid, piroxicam and felbinac are often used. Ibuprofen, naproxen, ketoprofen, etofenamate, aspirin and flufenamic acid are frequently used.

Moreover, so-called “natural” anti-inflammatory agents are useful in methods of the present invention. Such agents may suitably be obtained as an extract by suitable physical and/or chemical isolation from natural sources (e.g., plants, fungi, by-products of microorganisms) or can be synthetically prepared. For example, candelilla wax, bisabolol (e.g., alpha bisabolol), aloe vera, plant sterols (e.g., phytosterol), Manjistha (extracted from plants in the genus Rubia, particularly Rubia Cordifolia), and Guggal (extracted from plants in the genus Commiphora, particularly Commiphora Mukul), kola extract, chamomile, red clover extract, and sea whip extract, may be used.

Additional anti-inflammatory agents useful herein include compounds of the Licorice (the plant genus/species Glycyrrhiza glabra) family, including glycyrrhetic acid, glycyrrhizic acid, and derivatives thereof (e.g., salts and esters). Suitable salts of the foregoing compounds include metal and ammonium salts. Suitable esters include C₂-C₂₄ saturated or unsaturated esters of the acids, or C₁₀-C₂₄, or C₁₆-C₂₄. Specific examples of the foregoing include oil soluble licorice extract, the glycyrrhizic and glycyrrhetic acids themselves, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, and disodium 3-succinyloxy-beta-glycyrrhetinate. Stearyl glycyrrhetinate is preferred.

Specifically, the non-steroidal anti-inflammatory (NSAID) drug can selected from the group of aspirin, meclofenamate sodium, oxyphenbutazone, phenylbutazone, indomethacin, piroxicam, sulindac and tolmetin; mefenamic acid and zomepirac; ibuprofen, fenoprofen and naproxen.

The non-steroidal anti-inflammatory agent can be present in any suitable, safe and effective amount. For example, the non-steroidal anti-inflammatory agent can be present in up to about 5.0 wt. % of the composition of the present invention, up to about 2.0 wt. % of the composition of the present invention, or up to about 1.0 wt. % of the composition of the present invention.

### Biofilm-Dissolving Agent

Any suitable, effective and safe biofilm-dissolving agent can be employed in the composition of the present invention. For example, the biofilm-dissolving agent can be a furanone, protease, papain, N-acetylcysteine, mercaptoethanol, ebselen, L- and D-methionine, DNAse, RNAse, trypsin, sodium thiosulfate, glutathione, glutathione diethyl ester, fosfomycin, WR-2721, or any combination thereof. Specifically, the biofilm-dissolving agent can be N-acetyl cysteine (NAC).

The biofilm-dissolving agent can be present in the composition of the present invention in any suitable, safe and effective amount. For example, biofilm-dissolving agent can be present in the composition of the present invention in up to about 10.0 wt. % of the pharmaceutical composition, in up to about 5.0 wt. % of the pharmaceutical composition, or in up to about 2.0 wt. % of the pharmaceutical composition.

### Preservative

The composition of the present invention can optionally include a preservative. Any suitable, safe and effective preservative can be employed. Suitable preservatives include, e.g., benzalkonium chloride, quat-15, parabens, dichlorobenzyl alcohol, ethylene diamine tetraacetic acid (EDTA), disodium ethylene diamine tetraacetic acid (EDTA), formaldehyde, gum benzoin, imidazolidinyl urea, phenyl-mercuric acetate, poly aminopropyl biguanide, propyl gallate, sorbic acid, cresol, chloroacetamide sodium benzoate, chloromethyl-methylisothiazolinone, chloromethyl-methylisothiazolon, chloromethyl-methylisothiazolinone benzalkonium chloride, an octylisothiazolinone benzimidazole-compound, chloromethyl-methylisothiazolinone octylisothiazolinone, o-phenylphenol benzisothiazolinone, o-phenylphenol benzisothiazolinone, benzisothiazolinone, an aliphatic amine of 2-thiopyridineoxide, benzoic acid, editic acid, phenolic acid, benzyl alcohol, isopropyl alcohol, benzenethonium chloride, bronopol, cetrimide, chlorhexidine, chlorobutanol, chlorocresol, phenol, phenoxyethanol, phenyl ethyl alcohol, phenylmercuric acetate, phenylmercuric borate, phenylmercuric nitrate, potassium sorbate, proplyene glycol, sodium benzoate, sodium propionate, thimerosal, grapeseed extract and combinations thereof. Specifically, the preservative can be disodium EDTA, benzalkonium chloride, and combinations thereof.

The preservative can be present in the composition of the preset invention in any suitable, safe and effective amount. For example, preservative can be present in the composition of the present invention in up to about 5.0 wt. % of the pharmaceutical composition, in up to about 2.0 wt. % of the pharmaceutical composition, or in up to about 1.0 wt. % of the pharmaceutical composition.

### Pharmaceutical Formulations

The pharmaceutical compositions of the invention can be formulated with conventional carriers and excipients, which will be selected in accord with ordinary practice. Aqueous formulations are prepared in sterile form, and when intended for delivery by other than oral administration generally will be isotonic. All formulations will optionally contain excipients such as those set forth in the Handbook of Pharmaceutical Excipients, 5^{th} Ed.; Rowe, Sheskey, and Owen, Eds.; American Pharmacists Association; Pharmaceutical Press: Washington, D.C., 2006. Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 6.5 to about 8.5.

The formulations are suitable for the foregoing administration routes. The formulations may conveniently be presented in unit dosage form and may be prepared by any of the methods well known in the art of pharmacy. Techniques and formulations generally are found in Remington's Pharmaceutical Sciences, Mack Publishing Company, Easton, Pa., (1985). Such methods include the step of bringing into association the active ingredient(s) with the carrier, which constitutes one or more accessory ingredients. In general the formulations are prepared by uniformly and intimately bringing into association the active ingredient(s) with liquid carriers or finely divided solid carriers or both, and then, if necessary, shaping the product.

Specifically, the compositions of the invention can be formulated as a solution (e.g., ototopical solution), suspension, lotion, cream, ointment, aerosol, spray, gel or powder. More specifically, the compositions of the invention can be formulated as an ear drop.

### Routes of Administration

The compositions of the invention (herein referred to as the active ingredients) are administered by any route appropriate to the condition to be treated. Suitable routes include oral, nasal, topical (including buccal and sublingual), aural and parenteral (including subcutaneous, intramuscular, intravenous, intradermal, intrathecal and epidural), and the like. It will be appreciated that the preferred route may vary with for example the condition of the recipient.

Pharmaceutical kits useful in the present invention, which include a therapeutically effective amount of a pharmaceutical composition that includes a compound of component (a) and one or more compounds of component (b), in one or more sterile containers, are also within the ambit of the present invention. Sterilization of the container may be carried out using conventional sterilization methodology well known to those skilled in the art. Component (a) and component (b) may be in the same sterile container or in separate sterile containers. The sterile containers or materials may include separate containers, or one or more multi-part containers, as desired. Component (a) and component (b), may be separate, or physically combined into a single dosage form or unit as described above. Such kits may further include, if desired, one or more of various conventional pharmaceutical kit components, such as for example, one or more pharmaceutically acceptable carriers, additional vials for mixing the components, etc., as will be readily apparent to those skilled in the art. Instructions, either as inserts or as labels, indicating quantities of the components to be administered, guidelines for administration, and/or guidelines for mixing the components, may also be included in the kit.

All publications, patents, and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention. The present invention can be illustrated by the following non-limiting examples.

### EXAMPLES

### Example 1

VS is a 55 year old woman with a complicated otologic history. She contracted otitis media complicated by meningitis in 1988, and underwent bilateral tympanostomy tube (TT) placements at the time. She sustained two prior episodes of labyrinthitis in the left ear in 1987, and underwent a fistula repair that reduced her vertigo for a few years.

She developed vertigo and bilateral otalgia in 1997, and presented to our practice. Given her prior history, she underwent an extensive workup from the outset. Her MRI and CT of the temporal bones were essentially negative, and her ENG demonstrated a 46% left sided weakness, as would be expected from her prior episodes of labyrinthitis. She underwent right-sided T tube placement for chronic Eustachian tube dysfunction in October 1997, with marked improvement of her pressure and fullness. However, she developed intermittent otorrhea, for which she received topical Tobradex. In November, the tube plugged completely, and she underwent tube replacement. Findings were notable for thick mucus filling the middle ear space. She continued to drain through December despite aggressive topical therapy with Tobradex and ciprofloxacin drops. She underwent a right sided mastoidectomy with tube exchange in December 1997, with intraoperative findings notable for an inflamed mastoid cavity with several areas of cholesterol granuloma, as well as complete aditus blockade. Pathology from the surgery was benign, and cultures demonstrated only rare coagulase-negative Staphylococcus.

She continued to have thick, “taupe”-colored otorrhea after surgery. Considering her failure to respond to therapy, the differential was widened. Her evaluations for immunodeficiency and autoimmune pathology were negative. She underwent multiple TT placements and exchanges, as well as a negative culture and a wide laser tympanotomy in June 1998. The tympanotomy afforded her a brief hiatus from drainage from July through August. However, she soon redeveloped the same thick, purulent otorrhea, which failed to respond to Tobradex, chloramphenicol or ciprofloxacin drops, and Biaxin. She also required office debridements every week or alternate week until she underwent a revision tympanomastoidectomy in October 1998. Intraoperative findings were notable for a clear mastoid, but a middle ear full of thick mucopurulence and edematous mucosa. The KTP laser was used to widen the tympanotomy and to ablate the middle ear mucosa to the Eustachian tube. Intraoperative cultures returned negative. Her course improved considerably, with only mild intermittent infections through July 1999. However, she was then diagnosed with breast cancer, which was treated with a modified radical mastectomy and adjuvant chemoradiation.

As she was completing her chemotherapy, she redeveloped the same otorrhea, which was treated with continuous, suppressive Floxin drops as well as frequent office debridements. She developed a mastoid abscess in March 2000, for which she underwent a repeat revision mastoidectomy. Preoperative CT imaging raised the concern for osteomyelitis. Intraoperative findings were notable for purulence in the mastoid and subcutaneous tissues, which were debrided widely. Intraoperative cultures returned penicillin-resistant, but otherwise pan-sensitive Staphylococcus aureus. Her otorrhea resumed in April after a brief hiatus. She was maintained on Floxin drops until October, at which point she was started on topical vancomycin with some improvement. She continued to require office debridements on alternate weeks.

In April 2001, her otorrhea resolved after she was switched to ciprofloxacin drops. She had brief episodes of otorrhea in November 2001, and February and May 2002, which were treated easily with ototopicals.

Her otorrhea restarted in February 2003. She was begun on alternating courses of topical ciprofloxacin and Tobradex, oral azithromycin and metronidazole, as well as office debridements every week or alternating week. Her MRI in August 2003 demonstrated only scattered areas of hyperintensity through the mastoid. She had rare weeks without drainage, but her symptoms continued essentially unabated through November 2003. At this point, cultures of the otorrhea demonstrated two separate strains of MRSA. An infectious disease consultation was obtained, and she was started on high dose Septra per recommendations. She had minor improvement with the Septra, as well as a one-month course of Levaquin in August 2004, but continued to drain through November 2004. In the interim, she had 7 cultures demonstrating coagulase-negative Staphylococcus, diphtheroids, and Corynebacterium and Bacillus species. She also had one culture in September 2004 demonstrating 4+MRSA, which prompted a switch from oral Septra to Zyvox per the infectious disease consultant. A followup culture in October was negative.

Given her continued otorrhea, she underwent a revision modified radical tympanomastoidectomy in November 2004, with intraoperative findings notable for a large cholesterol granuloma extending from the mastoid tip to the retrosigmoid region towards the occiput. She had frank purulence in the epi-, pro-, and hypotympanum. The middle ear was packed with Zyvox-soaked Gelfoam. Intraoperative tissue cultures and follow-up clinic cultures proved negative. After a brief respite, her otorrhea restarted the following month. She failed to respond to topical Ciprodex and Volsol. Multiple cultures returned negative except for occasional colonies of Candida albicans. Her course continued essentially unchanged through November 2005.

Given her multiple treatment failures, she was started at this point on Ciprodex augmented with 2% NAC. Verbal informed consent was obtained prior to starting treatment, and she was continued on oral Zyvox. One week later, her nighttime otorrhea resolved, and the mucus in her middle ear was considerably thinner. Three weeks after starting treatment, both her otorrhea and middle ear mucus were fully resolved. At two months, she redeveloped a small amount of middle ear purulence, but was noted to be using drops that were about one month old. She was given a fresh mixture of NAC with Ciprodex, and her otorrhea and middle ear fluid resolved one week later. Two weeks later, she again developed middle ear purulence after using old drops, which were replaced. Cultures at this time demonstrated one colony of Sporobolomyces salmonicolor, but no bacterial growth. She was started on oral Lamisil for a presumed fungal superinfection, but could tolerate only three days of treatment secondary to gastrointestinal side effects. Her purulence again resolved, and she has remained drainage free for over one month with regular drop changes. She has been refrigerating the medication between uses, and hand warming the solution before application.

### Example 2

SG is a 59 year old diabetic woman with a history of frequent ear infections as a child. She developed ear plugging in May 2004, and was treated with oral antibiotics for otitis media. She then underwent right sided tympanostomy tube (TT) placement in June when her effusion failed to clear. However, she developed chronic otorrhea after tube placement, which persisted despite multiple courses of oral and topical antibiotics. The TT was removed in September, after which she developed a chronic serous effusion. She underwent a complete mastoidectomy and T tube placement in October, with intraoperative findings being notable for a very sclerotic mastoid and complete aditus blockade. The drainage restarted in December after a two month hiatus. Despite one course of oral Flagyl, continuous topical Tobradex, and intermittent CSF powder, the otorrhea persisted through February 2005. Her evaluations for Wegener's granulomatosis and cerebrospinal fluid leakage proved negative. Cultures of the drainage grew Candida albicans, which was treated with oral Ketoconazole and topical Volsol HC. She was continued on Volsol HC through March, at which point she developed a mild otitis externa. The T tube was removed, CSF powder was applied, and she was placed on dry ear precautions. However, she redeveloped a middle ear effusion by April, at which point a TT was replaced with return of a thin serous effusion. Her ear continued to drain through November despite intermittent topical Tobradex and oral Levaquin. Her drainage was recultured in September with no growth.

In November 2005, SG underwent a two week course of topical Ciprodex augmented with 2% NAC. Verbal informed consent was obtained prior to starting treatment. Her otorrhea resolved completely, and she had no signs of effusion. She required two additional 3 day courses of Ciprodex with 2% NAC for minimal crusting around the TT, but she has remained free of drainage through January 2006. Her hearing has remained stable by serial audiometry.

### Example 3

BT is a 47 year old man with diabetes and chronic Eustachian tube dysfunction. He sustained a sudden left-sided sensorineural hearing loss in February 2005, making optimization and preservation of his left sided hearing paramount. He also has a history of chronic sinusitis and polyposis, which is being managed with Nasonex, Astelin, Singulair, and nasal saline irrigations. He underwent bilateral functional endoscopic sinus surgery in April 2005.

He developed chronic serous otitis media in July 2005. A temporal bone CT scan demonstrated extensive middle ear and mastoid effusions. Bilateral T tubes were placed two weeks later after the effusions failed to resolve spontaneously. Both ears were noted to have thick, tenacious, mucoid effusions during the procedure. He was placed on ciprofloxacin otic drops, but developed recurrent otorrhea on the right side. The right sided tube was changed to a larger Activent tube to facilitate drainage and medication access. He was also placed on oral Levaquin for one month and continuous topical Ciprodex, but he continued to drain until September. However, his canal skin became quite macerated from chronic drop usage, and the Ciprodex was discontinued. He was treated intermittently with CSF powder through December, by which time he redeveloped a thick, right sided effusion behind a plugged tube. The tube was disimpacted, and he was again treated with topical Ciprodex and oral Augmentin, which cleared the effusion. However, he redeveloped a similar effusion in January 2006 after an upper respiratory tract infection.

At this point, BT was started on a two week course of topical Ciprodex augmented with 2% NAC. Verbal informed consent was obtained prior to starting treatment. H is effusion resolved, and his TT was clean and patent. H is conductive hearing loss improved slightly, and he shows no signs of ototoxicity.

### Example 4

DL is a 57 year old woman with Samter's triad and chronic right sided Eustachian tube dysfunction. She developed fullness and plugging of her right ear in January 2002 after a prolonged plane flight. She underwent TT placement in May, with return of a thick, mucoid effusion. The tube extruded prematurely, and she underwent T tube placement in June. A similar effusion was again noted. The effusion and plugging continued despite oral azithromycin and Sudafed, as well as continuous topical ciprofloxacin and Tobradex. By December, her tube extruded and her tympanic membrane perforation and otorrhea resolved. However, she redeveloped a similar effusion by March 2003, and another TT was inserted. This promptly plugged, and she underwent T tube insertion in April. Her tube plugging and otorrhea continued despite topical ciprofloxacin and Tobradex. She underwent a complete mastoidectomy and T tube replacement in August, with intraoperative findings being notable for a well pneumatized mastoid, thick effusion in both the mastoid and middle ear, and several areas of cholesterol granuloma. However, her drainage and tube plugging continued, with cultures in October demonstrating 1+yeast. She underwent clinic debridement and Activent tube placement in January 2004, and was started on topical Cipro HC. Her course continued essentially unchanged through November 2005, with debridements every 1-2 months and administration of various medications including Cipro HC, Ciprodex, Volsol HC, Floxin, azithromycin, and Augmentin.

In November 2005, DL was started on a two week course of topical Ciprodex augmented with 2% NAC. Verbal informed consent was obtained prior to starting treatment. However, minimal change was noted one month later. The same solution was insufflated into the ear after an aggressive debridement, and she was started on another two week course. The patient traveled abroad for one month, and returned in January 2006. Due to noncompliance issues, the patient's treatment was unsuccesful.

### Example 5

The following off-label mixture was prepared:

| | |
|---|---|
| 20% N-acetylcysteine | 0.17 cc |
| Ciprodex otic | 1.5 cc |
| Total volume | 1.67 cc |

Final Drug Concentrations:

| | |
|---|---|
| N-acetylcysteine | 2.04% |
| Ciprofloxacin | 0.27% |
| Dexamethasone | 0.090% |
| Disodium EDTA | >0.0051% |
| Benzalkonium chloride | 0.0090% |

Three to five drops were administered to the affected ear(s) two to three times daily for the durations specified above.

### Example 6

Active Ingredients:

| | |
|---|---|
| N-acetylcysteine | 2% |
| Ciprofloxacin | 0.3% |
| Dexamethasone | 0.1% |
| Disodium EDTA | 0.075% |
| Benzalkonium chloride | 0.01% |
| pH | 7.4 |

Inactive Ingredients:

Sodium chloride
Acetic acid
Sodium acetate
Hydroxyethyl cellulose
Tyloxapol (surfactant)
Boric acid
Purified water

The solution has been refrigerated at 2-4 degrees Celsius until dispensed. The solution may then be stored at room temperature between uses, with the knowledge that the solution will degrade within one month. The standard treatment course would include 3-5 drops to the affected ear(s) 2-3 times daily for 10-14 days.

1. A pharmaceutical composition comprising:
(a) at least one of a non-aminoglycoside antibiotic and an anti-inflammatory agent; and
(b) a biofilm-dissolving agent.

2. The pharmaceutical composition of claim 1, which is in the form of a solution, suspension, lotion, cream, ointment, aerosol, spray, gel or powder.

3. The pharmaceutical composition of claim 1, which is in the form of an ototopical solution.

4. The pharmaceutical composition of claim 1, which is in the form of an ear drop.

5. The pharmaceutical composition of claim 1, wherein the non-aminoglycoside antibiotic is a quinolone antibiotic.

6. The pharmaceutical composition of claim 1, wherein the non-aminoglycoside antibiotic is ciprofloxacin or ofloxacin.

7. The pharmaceutical composition of claim 1, wherein the non-aminoglycoside antibiotic is ciprofloxacin, present in up to about 1.0 wt. % of the pharmaceutical composition.

8. The pharmaceutical composition of claim 1, wherein the anti-inflammatory agent is a steroidal anti-inflammatory drug.

9. The pharmaceutical composition of claim 1, wherein the anti-inflammatory agent is a non-steroidal anti-inflammatory drug (NSAID).

10. The pharmaceutical composition of claim 1, wherein the anti-inflammatory agent is dexamethasone.

11. The pharmaceutical composition of claim 1, wherein the anti-inflammatory agent is dexamethasone, present in up to about 1.0 wt. % of the pharmaceutical composition.

12. The pharmaceutical composition of claim 1, further comprising a preservative.

13. The pharmaceutical composition of claim 1, further comprising a preservative selected from the group of disodium EDTA, present in up to about 1.0 wt. % of the pharmaceutical composition; benzalkonium chloride, present in up to about 0.10 wt. % of the pharmaceutical composition; and combinations thereof.

14. A method of killing or inhibiting the growth of a fungus, the method comprising contacting the fungus with the composition of claim 1, in an amount and for a period of time effective to kill or inhibit the growth of the fungus.

15. The method of claim 14, wherein the fungus is selected from Aspergillus flavus, Aspergillusfumigatus, Aspergillus niger, Candida albicans, Bipolaris sp., Curvularia sp., Exserohilum sp., Alternaria sp., Drechslera sp., Helminthosporium sp., Fusarium sp., Mucor sp. and combinations thereof.

16. A method of killing or inhibiting the growth of a virus, the method comprising contacting the virus with the composition of claim 1, in an amount and for a period of time effective to kill or inhibit the growth of the virus.

17. A method of killing or inhibiting the growth of a bacteria, the method comprising contacting the bacteria with the composition of claim 1, in an amount and for a period of time effective to kill or inhibit the growth of the bacteria.

18. The method of claim 17, wherein the bacteria is selected from the group of Staphylococcus aureus, Pseudomonas aeruginosa, Staphylococcus epidermidis, Pseudomonas sp., Proteus sp., Escherichia coli, Klebsiella sp., Streptococcus alpha-haemolyticus, Bacillus sp., Streptococcus faecalis, Streptococcus beta-haemolyticus, Escherichia alkal. Dispar, Streptococcus pneumoniae, Haemophilus sp., Acinetobacter sp., Alcaligenesfaecalis, Moraxella sp., Serratia sp., Actinobacter sp., Mycoplasma sp. and combination thereof.

19. The method of claim 14, wherein the fungal infection is associated with an infection of an ear, eye, respiratory tract, alimentary tract, skin bone, soft tissue, genitourinary tract, or combination thereof, in a mammal.

20. The method of claim 19, wherein the infection is associated with a biofilm-based infection, acute otitis media, chronic otitis media, chronic serous otitis media, recurrent acute otitis media, glue ear, acute mastoiditis, chronic mastoiditis, otitis extema, a biofilm-based infection on a prostheses (tympanostomy tubes, cochlear implants, ossicular prostheses, ocular implants; contact lenses; tracheal implants including tracheostomy tubes and tracheo-esophageal puncture tubes; dental implants; stents; orthopedic implants; urinary catheters; ureteral stents), otorrhea relating to a pressure equalization tube, cholesteatoma, petrous apicitis, an infection of a mucosal surface, a nasal infection, a paranasal sinus infection, ophthalmic infection, infection of Eustachian tube, an infection in the perioperative setting; conjunctivitis; scleritis; styes; acute sinusitis, chronic sinusitis; pneumonia; bronchitis; cystic fibrosis; empyema; pericarditis; a dental infection; cellulitis; impetigo; osteomyelitis; a urinary tract infection; pyelonephritis; cystitis, or any combination thereof.
